(19) United States
(12) Patent Application Publication
(10) Pub. No.: US 2004/0197934 A1
(43) Pub. Date: Oct. 7, 2004

(54) System for performing median partitioning as a method for diversity selection and identification of biologically active compounds

(76) Inventors: Jurgen Bajorath, Lynnwood, WA (US); Jeffrey W. Godden, Seattle, WA (US)

Correspondence Address: Michael L. Goldman, Esq., Nixon Peabody LLP, Clinton Square, P.O. Box 31051, Rochester, NY, 14603-1051, US

(21) Appl. No.: 10/760,988

(22) Filed: Jan. 20, 2004

Related U.S. Application Data
(60) Provisional application No. 60/441,341, filed on Jan. 17, 2003.

(51) Int. Cl. G06F 19/00; G01N 33/48; G01N 33/50; G01N 33/543

(52) U.S. Cl. 436/518; 702/19

(57) ABSTRACT

A system and method for identifying a small group of compounds representative of a larger set of compounds is disclosed. The system obtains one or more descriptors, determines the median value for the values of each descriptor for a set of compounds, partitions the set of compounds into a plurality of partitions using each median value for the set of compounds, and selects compounds from each of the partitions to form a subgroup representative of the set of compounds. A system and method for virtual compound screening is also disclosed. The system recursively partitions a set of compounds based on descriptor median values where the partitions which have at least two bait compounds are recombined and repartitioned until a desired number of compounds remain in the partition.

This application claims the benefit of U.S. Provisional Patent Application Serial No. 60/441,341 filed on Jan. 17, 2003, which is incorporated herein by reference in its entirety.

### FIELD OF THE INVENTION

This invention relates generally to computational chemistry and, more particularly, to systems and methods for selecting representative or diverse subsets from large compound database collections, the classification of compounds according to biological activity, and for virtual screening.

### BACKGROUND OF THE INVENTION

The selection of subsets from large compound pools, such as combinatorial libraries, inventories, or collections from vendor catalogs, is an important topic in molecular diversity analysis, for example, when developing compound acquisition strategies (Shemetulskis et al., “Enhancing the Diversity of a Corporate Database Using Chemical Database Clustering and Analysis,” J. Comput-Aided Mol. Des. 9:407-416 (1995); and Rhodes et al., “Bit-String Methods for Selective Compound Acquisition,” J. Chem. Inf. Comput. Sci. 2000, 40:210-214).

Major efforts in diversity analysis include subset selection and diversity design (Willett, “Dissimilarity-Based Algorithms for Selecting Structurally Diverse Sets of Compounds,” J. Comput. Biol. 6:447-457 (1999)). By definition, subset selection starts from given compound data sets and is in essence a deductive approach, whereas the design of diverse libraries is more inductive in nature. Various methods have been introduced to facilitate the selection of representative or diverse subsets from compound collections.

Prominent among those are clustering techniques (Willett, “Similarity and Clustering in Chemical Information Systems;” Research Studies Press; Letchworth (1987); Barnard et al., “Clustering of Chemical Structures on the Basis of Two-Dimensional Similarity Measures,” J. Chem. Inf. Comput. Sci. 32:644-649 (1992)), especially hierarchical clustering (Ward, “Hierarchical Grouping to Optimize an Objective Function,” J. Am. Stat. Assoc., 58:236-244 (1963)), stochastic methods combining different diversity functions and search algorithms, (Agrafiotis, “Stochastic Algorithms for Maximizing Molecular Diversity,” J. Chem. Inf. Comput. Sci. 37:841-851 (1997)) and dissimilarity-based methods, (Willett, “Dissimilarity-Based Algorithms for Selecting Structurally Diverse Sets of Compounds,” J. Comput. Biol. 6:447-457 (1999); Snarey et al., “Comparison of Algorithms for Dissimilarity Based Compound Selection,” J. Mol. Graph. Model. 15:372-285 (1997)), which include, among others, different versions of the popular MaxMin algorithm. (Higgs et al., “Experimental Designs for Selecting Molecules From Large Chemical Databases,” J. Chem. Inf. Comput. Sci. 37:861-870 (1997); Clark, “OptiSim: An Extended Dissimilarity Selection Method for Finding Diverse Representative Subsets,” J. Chem. Inf. Comput. Sci. 37:1181-1188 (1997)).

Like molecular fingerprint-based approaches in diversity selection (Shemetulskis et al., “Stigmata: An Algorithm to Determine Structural Commonalities in Diverse Datasets,” J. Chem. Inf. Comput. Sci. 36:862-871 (1996); Xue et al, “A Dual-Fingerprint Based Metric for the Design of Focused Compound Libraries and Analogues,” J. Mol. Model. 7:125-131 (2001)), these techniques essentially rely on pairwise comparisons of property distances between compounds. In principle, diversity functions that rely on pairwise molecular comparisons display quadratic dependence on the number of compounds in the data set. In consequence, the underlying combinatorial problem substantially increases with the size of both databases and subsets and becomes computationally infeasible if the data sets are very large.

Different types of dissimilarity-based methods with modulated complexity have been developed (Willett, “Dissimilarity-Based Algorithms for Selecting Structurally Diverse Sets of Compounds,” J. Comput. Biol. 6:447-457 (1999)). For example, the complexity of maximum dissimilarity selection methods is on the order of O(kn) to O(k²n), with k being the size of the subset and n the size of the original collection. More efficient techniques for diversity analysis, such as the centroid-based diversity sorting algorithm (Holliday et al., “Fast Algorithm for Selecting Sets of Dissimilar Molecules From Large Chemical Databases,” Quant. Struct. Act. Relat,” 14:501-506 (1995)), have been introduced where complexity only scales with the size of the original data set and for which further improvements in calculation speed have recently been proposed (Trepalin et al., “New Diversity Calculations Algorithms Used for Compound Selection,” J. Chem. Inf. Comput. Sci., 42:249-258 (2002)). In addition, other algorithms have been designed that rely on probability sampling rather than complete enumeration of pairwise distances (Agrafiotis, “A Constant Time Algorithm for Estimating the Diversity of Large Chemical Libraries,” J. Chem. Inf. Comput. Sci. 41:159-167 (2001)) and thereby largely circumvent the combinatorial problem.

Cell-based methods represent a different approach for compound classification and selection to partition compound data sets because they do not depend on distance or nearest neighbor calculations (Cummins et al, “Molecular Diversity in Chemical Databases: Comparison of Medical Chemistry Knowledge Bses and Databases of Commercially Available Compounds,” J. Chem. Inf. Comput. Sci. 36:750-763 (1996); Pearlman et al., “Novel Software Tools for Chemical Diversity,” Perspect. Drug Discov. Design 9:339-353 (1998); Xue et al, “Molecular Descriptors for Effective Classification of Biologically Active Compounds Based on Principal Component Analysis Identified by a Genetic Algorithm,” J. Chem. Inf. Compu. Sci., 40:801-809 (2000)).

Cell-based methods involve calculating positions of molecules in low-dimensional property spaces and identifying the cells into which compounds fall. Cells are subdivisions of chemical space obtained by application of binning schemes. (Bayley et al., “Binning Schemes for Partition-Based Compound Selection,” J. Mol. Graph. Model. 17:10-18 (1999)). Similar to the situation in cluster analysis (Willett, “Similarity and Clustering in Chemical Information Systems,” Research Studies Press; Letchworth (1987)), representative compounds can then be selected from each computed cell. Since partitioning does not require calculation of pairwise property distances, the complexity of the methods is lower than in the case of clustering or maximum dissimilarity methods on the order of O(n) similar to centroid-based diversity sorting.

It follows that cell-based methods should, in principle, be amenable to the analysis of much larger compound pools than methods depending on pairwise comparisons. However, cell-based methods generally require a dimension reduction of chemical descriptor space (Pearlman et al., “Novel Software Tools for Chemical Diversity,” Perspect. Drug Discov. Design, 9:339-353 (1998); Xue et al., “Molecular Descriptors for Effective Classification of Biologically Active Compounds Based on Principal Component Analysis Identified by a Genetic Algorithm,” J. Chem. Inf Compu. Sci., 40:801-809 (2000)), which can be accomplished, for example, by principal component analysis (“PCA”) (Glen et al., “Principal Component Analysis and Partial Least Squares Regression,” Tetrahedron Comput. Methodol., 2:349-376 (1989)).

However, increasing the size of the original compound pool becomes an issue due to the increasing complexity of eigenvalue and eigenvector calculations when computing principal components (Glen et al., “Principal Component Analysis and Partial Least Squares Regression,” Tetrahedron Comput. Methodol. 2:349-376 (1989)). But, not all partitioning methods are cell-based. For example, recursive partitioning (Friedman, “Recursive Partitioning Decision Rules for Nonparametric Classification,” IEEE Trans. Comput., 26:404-408 (1997); Rusinko et al., “Analysis of a Large Structure/Biological Activity Data Set Using Recursive Partitioning,” J. Chem. Inf. Comput. Sci. 39:1017-1026 (1999)), which is mostly applied for hit or lead identification, generates subsets along decision trees.

Compound classification and virtual screening methods are capable of exploring and exploiting molecular similarity beyond chemistry, in accordance with the similar property principle (Johnson et al., Concepts and Applications of Molecular Similarity, New York: John Wiley & Sons (1990)). They can be used to analyze and predict biologically active compounds and correlate structural features and chemical properties of molecules with specific activities. This explains why such approaches are highly attractive tools in pharmaceutical research (Walters et al., “Virtual Screening-An Overview,” Drug Discovery Today 3:160-178 (1998)), although a number of the underlying scientific concepts have originally been developed for different purposes.

Since it is increasingly recognized that simply synthesizing and screening more and more compounds does not necessarily provide a sufficiently large number of high-quality leads and, ultimately, clinical candidates, much effort is spent in developing and implementing computational concepts that help to identify and refine leads. Typical applications include the identification of compounds with desired activity by database searching, derivation of predictive models of activity for database mining, selection of representative subsets from large compound libraries, or analysis of drug-like properties.

A prerequisite for most approaches to compound classification and library design or analysis is the definition of theoretical “chemical space.” Similar to qualitative structure-activity relationship (“QSAR”) investigations, this typically involves the use of descriptors that capture a broad range of molecular characteristics (Livingstone, “The Characterization of Chemical Structures Using Molecular Properties. A Survey,” J. Chem. Inf. Comput. Sci. 40:195-209 (2000); Xue et al., “Molecular Descriptors in Chemoinformatics, Computational Combinatorial Chemistry, and Virtual Screening,” Comb. Chem. High Throughput Screening 3:363-372 (2000)). Such molecular descriptors may have very different complexity but can often be classified according to their “dimensionality,” referring to the molecular representations from which they are calculated (Xue et al., “Molecular Descriptors in Chemoinformatics, Computational Combinatorial Chemistry, and Virtual Screening,” Comb. Chem. High Throughput Screening 3:363-372 (2000)).

The majority of conventional compound classification approaches are based on clustering (Barnard et al., “Clustering of Chemical Structures on the Basis of Two-Dimensional Similarity Measures,” J. Chem. Inf. Comput. Sci. 32:644-649 (1992)), or partitioning methods (Mason et al., “Partition-Based Selection,” Perspect. Drug Discovery Des., 7/8:85-114 (1997)). Clustering of compounds in chemical space, however defined, typically involves the calculation of intermolecular distances, and compounds that are “close” to each other are combined into clusters.

In partitioning, on the other hand, chemical space is subdivided into sections, based on ranges of descriptor values, and compounds that fall into the same section are combined. For compound partitioning, it is important how chemical space is divided into cells, and this process depends on the way descriptor value ranges are binned (Bayley et al., “Binning Schemes for Partition-Based Compound Selection,” J. Mol. Graphics Modell. 17:10-18 (1999)). Binning produces “cells” in chemical space, and the analysis of how these subspaces are populated with compounds is a common theme of cell-based partitioning methods (Pearlman et al., “Metric Validation and the Receptor-Relevant Subspace Concept,” J. Chem. Inf. Comput. Sci. 39:28-35 (1999); Barnard et al., “Clustering of Chemical Structures on the Basis of Two-Dimensional Similarity Measures,” J. Chem. Inf. Comput. Sci. 32:644-649 (1992); Mason et al., “Partition-Based Selection,” Perspect. Drug Discovery Des., 7/8:85-114 (1997)). Such approaches benefit from the ability to generate low-dimensional chemistry space.

A major goal of many compound classification studies is to select representative subsets of large libraries, for example, which mirror their overall diversity. Another attractive application is the selection of active compounds or the separation of active and inactive molecules. In the latter cases, the calculations attempt to produce clusters or cells that are enriched with molecules having desired activity or that contain only molecules with a specific activity, while minimizing the number of classes that mix compounds with different activities and the number of singletons (i.e., clusters or cells containing only one compound). Since the choice of calculation parameters and descriptors influences the number, size, and composition of clusters or cells, many investigations aim to identify combinations of algorithms and calculation conditions that optimally separate compounds in benchmark databases.

Virtual screening methods are designed for searching large compound databases in silico and selecting a limited number of candidate molecules for testing to identify novel chemical entities that have the desired biological activity (Bajorath, “Selected Concepts and Investigations in Compound Classification, Molecular Descriptor Analysis, and Virtual Screening,” J. Chem. Inf. Comput. Sci. 41:233-245 (2001)). Further, virtual screening is often discussed in the context of chemoinformatics (Brown, “Chemoinformatics: What Is It and How Does It Impact Drug Discovery,” Annu. Rep. Med. Chem. 33:375-384 (1998); Agrafiotis et al., “Combinatorial Informatics in the Post Genomics Era,” Nature Rev. Drug Discov. 1:337-346 (2002)). Its main origins are protein-structure-based compound screening or docking (Kuntz, “Structure-Based Strategies For Drug Design and Discovery,” Science 257:1078-1082 (1992); Halpering et al., “Principles of Docking: An Overview of Search Algorithms and a Guide To Scoring Functions,” Proteins 47:409-443 (2002)) and chemical-similarity searching based on small molecules (Willett et al., “Chemical Similarity Searching,” J. Chem. Inf. Comput. Sci. 38:983-996 ( 1998)).

Recursive partitioning (“RP”), for example, is a statistical method for analyzing and mining large data sets that consist of active and inactive molecules, which was adapted by Young, Rusinko and colleagues (Chen et al., “Recursive Partitioning Analysis of a Large Structure-Activity Data Set Using Three-Dimensional Descriptors,” J. Chem. Inf. Comput. Sci. 38:1054-1062 (1998); Rusinko et al., “Analysis of a Large Structure-Biological Activity Data Set Using Recursive Partitioning,” J. Chem. Inf. Comput. Sci. 39:1017-1026 (1999)). RP divides data sets along decision trees.

At every branch or node, single or multiple binary descriptors, such as structural fragments, atom-pair or topological descriptors, are selected to divide the data into sets of molecules that share or do not share these descriptors (Cho et al., “Binary Formal Inference-Based Recursive Modeling Using Multiple Atom and Physicochemical Property Class Pair and Torsion Descriptors as Decision Criteria,” J. Chem. Inf. Comput. Sci. 40:668-680 (2000)). This leads to enrichment of partitions with active molecules, which can be monitored, for example, by calculating the average biological activity at each node. Finally, structures of active molecules are associated with specific descriptor settings, which in turn can be applied as rules to search databases for compounds that have similar activity. However, this requires learning sets for predictive model building.

Thus, a need exists for an efficient and fast method to facilitate the selection of diverse subsets and for selecting representative subsets of compounds from large databases. Specifically, an approach is needed that does not depend on pairwise comparison of compounds and that can be applied to very large pools of, ultimately, millions of molecules. Yet another need is for an easy-to-apply method of searching for compounds having similar activity for classifying compounds according to biological activity with reasonably high classification accuracy. Still further, there is a need for virtual screening applications that can be directly applied and which do not require learning sets for predictive model building.

### SUMMARY OF THE INVENTION

The present invention relates to a system for identifying a small group of compounds representative of a larger set of compounds. The system includes a descriptor system, a median determination system, a partitioning system, and a partition selection system. The descriptor system obtains one or more descriptor values for information representing each compound in the set of compounds, and the median determination system determines a median value for each of the descriptor values for the set of compounds. The partitioning system partitions the set of compounds into a plurality of partitions using each median value for the set of compounds. The partition selection system may then select compounds from each of the partitions to form a subgroup representative of the set of compounds.

Another aspect of the system for identifying a small group of compounds representative of a larger set of compounds includes the partition selection system determining a partition median value for each of the descriptor values for the compounds within a partition and selecting from the partition one or more compounds that have each descriptor value being within a predetermined range of values away from a corresponding partition median value to represent the compounds within the partition.

The present invention also relates to a method and a program storage device that is readable by a machine and tangibly embodies a program of instructions that is executable by the machine to perform a method for identifying a small subgroup of compounds representative of a larger set of compounds. The method includes providing a set of compounds and obtaining one or more descriptor values for each compound in the set of compounds. A median value is determined for each of the descriptor values for the set of compounds and the set of compounds is partitioned into a plurality of partitions using each median value for the set of compounds. Compounds are then selected from each of the partitions to form a subgroup of compounds representative of the set of compounds.

Another aspect of the method and program storage device for identifyng a small subgroup of compounds representative of a larger set of compounds includes determining a partition median value for each of the descriptor values for the compounds within a partition, and selecting from the partition one or more compounds that have each descriptor value being within a predetermined range of values away from a corresponding partition median value to represent the compounds within the partition.

The present invention also relates to a system for virtual compound screening that includes a bait compound system, a descriptor system, a median determination system, a partitioning system, a partition recombination system, and a selection system. The bait compound system combines a plurality of unidentified compounds with information representing a plurality of bait compounds having known biological activities to form a set of compounds. The descriptor system obtains one or more descriptor values for each of the unidentified compounds and for each of the bait compounds in the set of compounds, and the median determination system determines a median value for each of the descriptor values for the set of compounds. The partitioning system partitions the set of compounds into a plurality of partitions based on each median value, and the partition recombination system then recombines partitions which have at least two bait compounds to form a recombined set of compounds. A selection system then selects the recombined set of compounds for analysis of biological activity if an approximate target number of unidentified compounds remain in the recombined set of compounds.

The present invention also relates to a method and a program storage device that is readable by a machine and tangibly embodies a program of instructions that is executable by the machine to perform a method for virtual compound screening. The method includes combining a plurality of unidentified compounds with a plurality of bait compounds having known biological activities to create a set of compounds. One or more descriptor values are obtained for each of the unidentified compounds and for each of the bait compounds in the set of compounds. A median value is obtained for each of the descriptor values for the set of compounds and the set of compounds are partitioned into a plurality of partitions based on each median value. Partitions which have at least two bait compounds are recombined to form a recombined set of compounds, and the recombined set of compounds is selected for analysis of biological activity if an approximate target number of unidentified components remain in the recombined set of compounds.

The present invention offers a number of advantages over conventional methods for the selection of representative or diverse subsets from large compound collections, the classification of compounds according to biological activity, and for virtual screening. For example, the invention provides an efficient and conceptually straightforward method to facilitate the selection of diverse subsets. Specifically, the approach does not depend on pairwise comparison of compounds and can be applied to very large pools of, ultimately, millions of molecules.

Another advantage of the present invention is its ability to efficiently generate subsets of targeted size from very large compound pools. The present invention also makes use of quartile selection so that there is less vulnerability to boundary effects. The present invention is also able to employ many different types of molecular descriptors. Furthermore, the present invention easily monitors the occupancy rates of partitions and different numbers of compounds can be detected from variably populated partitions to mirror the composition of source data sets. Yet another benefit provided by the present invention is that it is capable of classifying compounds according to biological activity with a reasonably high classification accuracy.

Still further, the present invention advantageously does not depend on learning sets to derive predictive models of activity. Furthermore, in contrast to popular cell-based partitioning approaches, which create low-dimensional chemistry space for compound classification, the present invention operates in n-dimensional descriptor space and does not involve dimension reduction or secondary manipulations, other than transforming each descriptor contribution into a binary classification scheme.

### BRIEF DESCRIPTION OF THE DRAWINGS

FIG. 1 is a block diagram of a system for identifying a small group of compounds representative of a larger set of compounds in accordance with one embodiment of the present invention;

FIG. 2 is a functional block diagram of the memory used in the system shown in FIG. 1;

FIG. 3 is a flow chart of a process for identifying a small group of compounds representative of a larger set of compounds in accordance with another embodiment of the present invention;

FIG. 4 is a diagram of a compound pool in accordance with an embodiment of the present invention;

FIG. 5 is a diagram showing exemplary molecular descriptor value distributions in accordance with embodiments of the present invention;

FIGS. 6-8 are diagrams of compound pools in accordance with an embodiment of the present invention;

FIGS. 9-10 are diagrams of genetic algorithm processes in accordance with embodiments of the present invention;

FIG. 11 is a functional block diagram of the memory used in the system shown in FIG. 1 in accordance with another embodiment of the present invention;

FIG. 12 is a flow chart of a process for virtual screening in accordance with yet another embodiment of the present invention; and

FIGS. 13-17 are diagrams of compound pools in accordance with an embodiment of the present invention.

### DETAILED DESCRIPTION OF THE INVENTION

The present invention relates to a system for identifying a small group of compounds representative of a larger set of compounds. The system includes a descriptor system, a median determination system, a partitioning system, and a partition selection system. The descriptor system obtains one or more descriptor values for information representing each compound in the set of compounds, and the median determination system determines a median value for each of the descriptor values for the set of compounds. The partitioning system partitions the set of compounds into a plurality of partitions using each median value for the set of compounds. The partition selection system may then select compounds from each of the partitions to form a subgroup representative of the set of compounds.

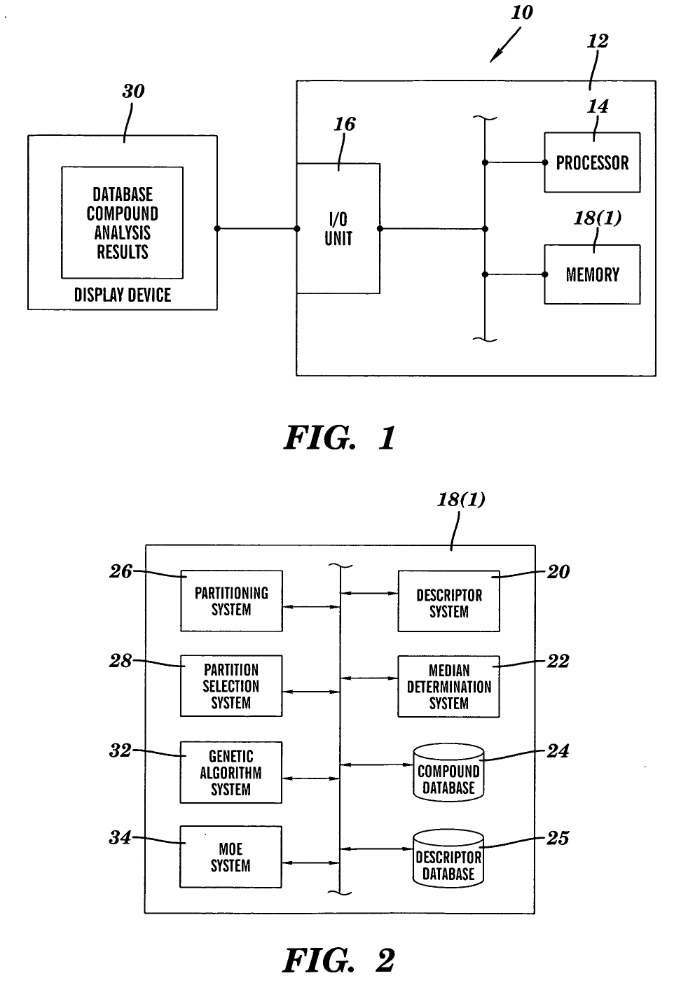

Referring to FIGS. 1 and 2, a system 10 that includes a computer 12 and a display device 30 is shown, although the system 10 can include a lesser or greater number of devices. The computer 12 and display device 30 are communicatively coupled to each other by a hard-wire connection over a local area network, although a variety of communication systems and/or methods using appropriate protocols can be used, including a direct connection via serial or parallel bus cables, a wide area network, the Internet, modems and phone lines, wireless communication technology, and combinations thereof.

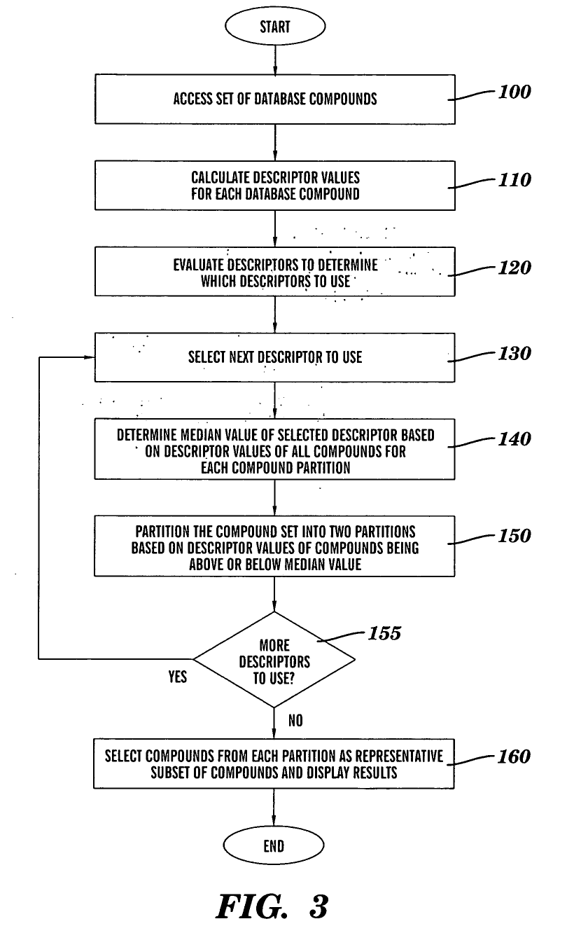

The computer 12 is provided for exemplary purposes only and may comprise other devices, such as a laptop or personal digital assistant. In the embodiments of the present invention, the computer 12 includes a processor 14, an I/O unit 16, a memory 18(1) and a user input system (e.g., keyboard and/or mouse) (not illustrated), which are coupled together by one or more bus systems or other communication links, although the computer 12 can comprise other elements in other arrangements. The processor 14 executes instructions stored in the memory 18(1) for identifying a small group of compounds representative of a larger set of compounds in accordance with at least one of the embodiments and examples of the present invention as described herein and which is illustrated in FIG. 3, although the processor 14 may perform other types of functions. The I/O unit 16 enables the computer 12 to communicate with the display device 30 by way of the hard-wire connection mentioned above.

The memory 18(1) comprises a variety of different types of memory storage devices, such as random access memory (“RAM”) or read only memory (“ROM”) in the computer 12, and/or a floppy disk, hard disk, CD-ROM or other computer readable medium which is read from and/or written to by a magnetic, optical, or other reading and/or writing system coupled to the processor 14. The memory 18(1) stores the instructions for identifying a small group of compounds representative of a larger set of compounds in accordance with at least one of the embodiments and examples of the present invention, although some or all of these instructions and data may be stored elsewhere.

In this particular embodiment, the memory 18(1) stores data and instructions, which when executed by the processor 14 as described further herein, implement a descriptor system 20, a median determination system 22, a compound database 24, a descriptor database 25, a partitioning system 26, a partition selection system 28, a genetic algorithm system 32, and a molecular operating environment (“MOE”) system 34, for identifying a small group of compounds representative of a larger set of compounds. The instructions for implementing these systems may be expressed as executable programs written in a number of conventional or later developed programming languages that can be understood and executed by the processor 14.

The descriptor system 20 comprises instructions stored in the memory 18(1), which when executed by the processor 14, evaluates the molecular property descriptors from the descriptor database 25 to determine the optimal set of descriptors to use for selecting diverse subsets of compounds, for example.

The median determination system 22 comprises instructions stored in the memory 18(1), which when executed by the processor 14, calculates median values for descriptor values of a set of compounds.

The compound database 24 comprises data representing a plurality of compounds from a variety of compound sources that are organized in the memory 18(1), such as the Available Chemicals Directory (“ACD”) (Available Chemicals Directory, MDL Information Systems, Inc., 14600 Catalina Street, San Leandro, Calif. 94577, which is hereby incorporated by reference herein in its entirety), although the compounds in the compound database 24 may originate from a variety of sources, such as from catalogs of various chemistry vendors. Further, the data representing each of the compounds in the compound database 24 describes a particular compound, such as the name of the compound and various properties of the compound.

The descriptor database 25 comprises data representing a plurality of molecular property descriptors organized in the memory 18(1). Each molecular property descriptor represents a numerical description for a particular property of a compound. Every descriptor has a unique name, or code, which identifies the descriptor and is used as a database field name in the descriptor database 25, for example.

Examples of molecular property descriptors include: a sum of atomic polarizabilities of all atoms; a number of aromatic atoms; a number of H-bond donors; a number of heavy atoms; a number of hydrophobic atoms; a number of nitrogen atoms; a number of fluorine atoms; a number of sulfur atoms; a number of iodine atoms; a number of bonds between heavy atoms; a number of aromatic bonds; a number of double nonaromatic bonds; an atomic connectivity index (order 0); a carbon valence connectivity index (order 1); a carbon connectivity index (order 1); a greatest value in a distance matrix; a third kappa shape index; a relative negative partial charge; a total positive van der Waals surface area; a fractional negative polar van der Waals surface area; a fractional hydrophobic van der Waals surface area; a vertex adjacency information (magnitude); a vertex distance equality index; a vertex distance magnitude index; a sum of a van der Waals surface area of each of one or more atoms in each compound in the set of compounds; a van der Waals surface area calculated for a property of each compound selected from the group consisting of hydrogen-bond acceptor atoms; hydrogen-bond donor atoms; nondonor-acceptor atoms; and polar atoms; a van der Waals volume calculated using a connection table; and a Zagreb index; molecular weight; and the number of atoms, although other descriptors could be used. Furthermore, a detailed description of a basic descriptor is disclosed by Xue et al., “Accurate Partitioning of Compounds Belonging to Diverse Activity Classes,” J. Chem. Inf. Comput. Sci. 42:757-764 (2002), which is hereby incorporated by reference in its entirety.

The partitioning system 26 comprises instructions stored in the memory 18(1), which when executed by the processor 14, partitions one or more sets of compounds into partitions based on median values of descriptor values for each of the compounds in the sets of compounds.

The partition selection system 28 comprises instructions stored in the memory 18(1), which when executed by the processor 14, selects one or more representative compounds from each of a plurality of partitions.

The genetic algorithm system 32 comprises instructions stored in the memory 18(1), which when executed by the processor 14, implements a genetic algorithm as described in Forrest, “Genetic Algorithms—Principles of Natural Selection Applied to Computation,” Science, 261:872-878 (1993), which is hereby incorporated by reference in its entirety.

The MOE system 34 comprises instructions stored in the memory 18(1), which when executed by the processor 14, implements the Molecular Operating Environment Version 2001.01 (Molecular Operating Environment, version 2001.01, Chemical Computing Group Inc., 1255 University Street, Montreal, Quebec, Canada, H3B 3X3, which is hereby incorporated by reference in its entirety). The processor 14 executes the instructions stored in the memory 18(1) that implement the MOE system 34 to calculate descriptor values for compounds.

The display device 30 comprises a computer monitor (e.g., CRT, LCD or plasma display device), although the display device 30 may comprise other types of display systems, such as a projection screen or a television. Further, the display device 30 is provided for exemplary purposes only and may comprise other information output devices, such as a printer. The display device 30 presents the results from execution by the processor 14 of the instructions stored in the memory 18(1). Since devices, such as the display device 30, are well known in the art, the specific elements, their arrangement within display device 30 and operation will not be described in further detail herein.

The present invention also relates to a method for identifying a small subgroup of compounds representative of a larger set of compounds. The method will now be described in the context of being carried out by the system 10 with reference to FIGS. 1-10. Basically, the method includes providing a set of compounds and obtaining one or more descriptor values for each compound in the set of compounds. A median value is determined for each of the descriptor values for the set of compounds and the set of compounds is partitioned into a plurality of partitions using each median value for the set of compounds. Compounds are then selected from each of the partitions to form a subgroup of compounds representative of the set of compounds.

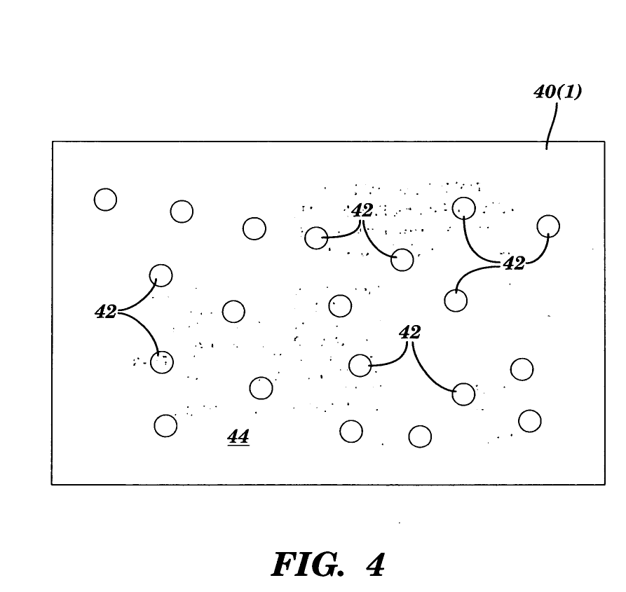

By way of example only, a user operating computer 12 desires selecting diverse subsets of compounds from the compound database 24. Referring to FIG. 3 and beginning at step 100, the user manipulates the input system of the computer 12 to send signals to the processor 14 that cause the processor to begin executing the instructions stored in the memory 18(1) which comprise the descriptor system 20. In response, the processor 14 accesses the compound database 24 to obtain a compound pool 40(1) comprising the database compounds 42 (based on all the compounds in the compound database 24) for further processing as described herein, although the database compounds 42 could be stored and obtained from other locations. It should be noted that only a portion of all the compounds obtained from the compound database 24 are illustrated in FIGS. 4 and 6-8. Further, the reference number (i.e., 42) in FIGS. 4 and 6-7 are shown as identifying just some of the database compounds 42 in the compound pools 40(1)-40(3) for clarity, but it should be understood that all of the transparent or unfilled circles in FIGS. 4 and 6-8 represent all of the database compounds 42 obtained from the compound database 24. It should also be noted that the compound pool 40(1) comprises an initial or first partition 44.

At step 110, the processor 14 executes the instructions stored in the memory 18(1) which comprise the descriptor system 20 and the MOE system 34 to calculate values for each of the descriptors from the descriptor database 25 for each of the database compounds 42 of the initial partition 44 in the compound pool 40(1). The processor 14 stores the calculated descriptor values in the memory 18(1) for further processing as described herein.

At step 120, the processor 14 executes the instructions stored in the memory 18(1) which comprise the descriptor system 20 to evaluate the descriptors for determining the optimal set of descriptors to use for selecting diverse subsets of database compounds 42 from the compound pool 40(1). Basically, the descriptor system 20 selects descriptors that will be suitable for calculating useful median values based on the particular database compounds 42 in the compound pool 40(1). To produce useful median values, the descriptors should yield “broad” or “information-rich” value distributions.

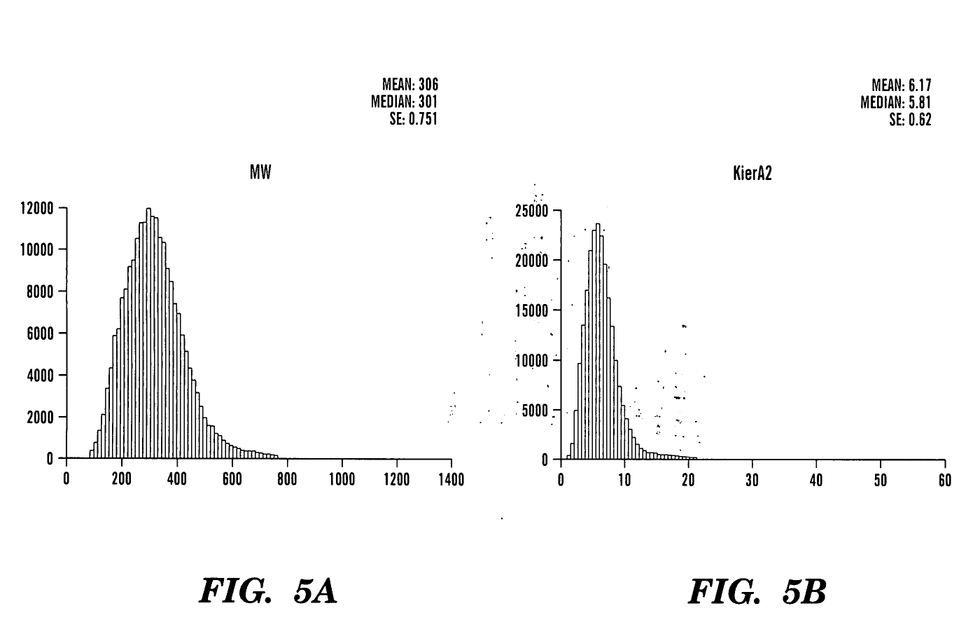

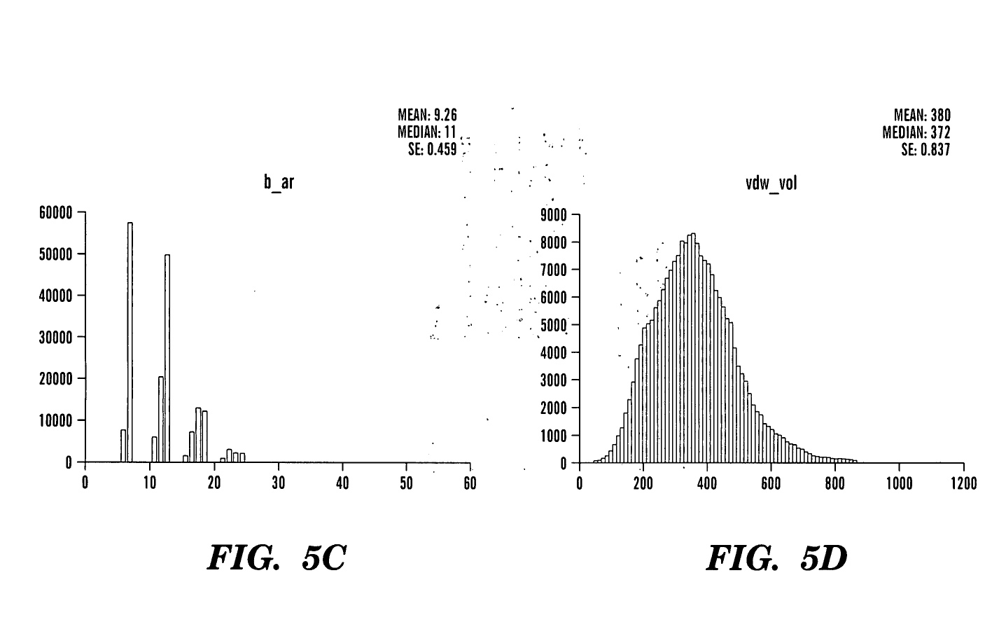

Referring to FIG. 5, exemplary value distributions of four arbitrary molecular descriptors (i.e., MW=molecular weight; b_ar=number of aromatic bonds; KierA2=Kier and Hall index; and vdw_vol=van der Waals volume) calculated for a total of 229,529 compounds from the Available Chemicals Directory (“ACD”) (Available Chemicals Directory, MDL Information Systems, Inc., 14600 Catalina Street, San Leandro, Calif. 94577, which is hereby incorporated by reference in its entirety) are shown. The value distributions shown in FIG. 5 are examples of some of the suitable or information-rich descriptors that can be used in the embodiments and examples of the present invention. Descriptor value distributions are monitored in histograms consistently having 100 bias, and mean, median and scaled SE values are reported for each descriptor (Godden et al., “Chemical Descriptors with Distinct Levels of Information Content and Varying Sensitivity to Differences Between Selected Compound Databases Identified by SE-DSE Analysis,” J. Chem. Inf. Comput. Sci., 42:87-93 (2002), which is hereby incorporated by reference in its entirety).

Additionally, the processor 14 executes the instructions stored in the memory 18(1) which comprise the descriptor system 20 to select information-rich descriptors that do not substantially correlate with each other. Identifying and selecting descriptors with as little correlation as possible avoids creating empty, under-populated and/or over-populated compound partitions at step 150. While it is difficult to identify information-rich descriptors with little or no correlation with each other, the processor 14 executes the instructions stored in the memory 18(1) which comprise the descriptor system 20 and the genetic algorithm system 32 to optimize descriptor combinations and minimize correlation effects. The processor 14 stores the descriptors that are identified as being information-rich while having the least amount of correlation with respect to each other in the memory 18(1) for further processing as described herein.

Here, the processor 14 executes the instructions stored in the memory 18(1) which comprise the descriptor system 20 to identify a plurality of information-rich descriptors that do not substantially correlate with each other for exemplary purposes only, but the user of the computer 12 desires using just two of the suitable descriptors (i.e., a first and a second suitable descriptor) and uses the input system of the computer 12 to cause the processor 14 to select the two suitable descriptors, although a lesser or greater number of suitable descriptors may be used.

At step 130, the processor 14 executes the instructions stored in the memory 18(1) which comprise the descriptor system 20 to select one of the two descriptors determined to be suitable for calculating useful median values at step 120 for further processing as described below in connection with step 140.

At step 140, the processor 14 executes the instructions stored in the memory 18(1) which comprise the median determination system 22 to calculate the median value of the descriptor selected above at step 130 based on the descriptor values of the selected descriptor for all of the database compounds 42 of the initial partition 44 in the compound pool 40(1) that are calculated at step 110. It is well known that a median is defined as the value within a value distribution that divides a population into two substantially equal subpopulations above and below the median value (Meier et al., “Statistical Methods in Analytical Chemistry,” John Wiley & Sons, New York (2000), which is hereby incorporated by reference in its entirety).

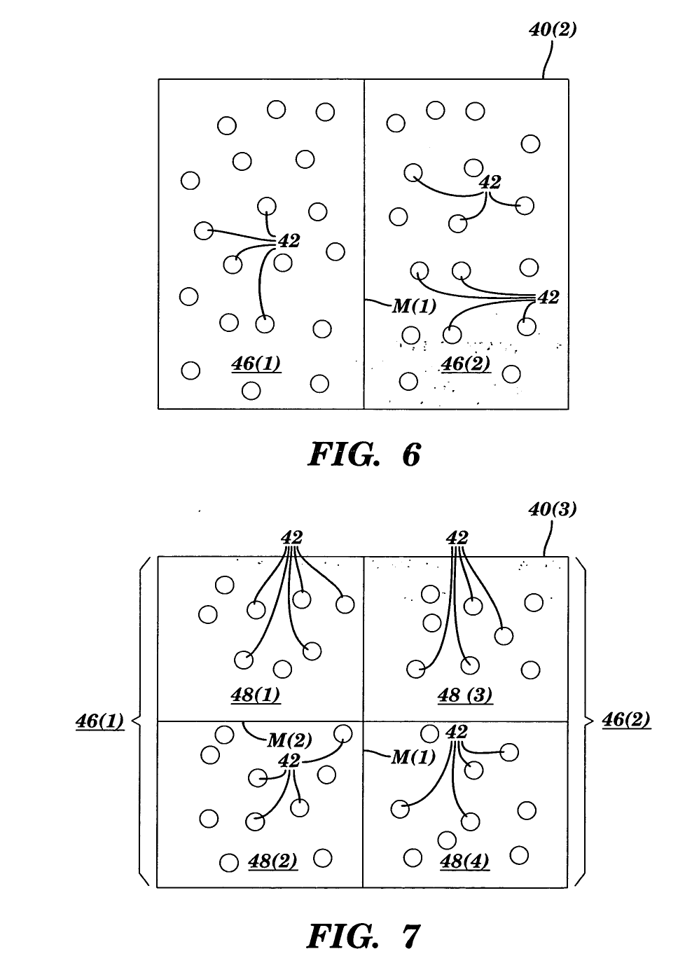

At step 150, the processor 14 executes the instructions stored in the memory 18(1) which comprise the partitioning system 26 to partition each partition (i.e., the initial partition 44) in the compound pool 40(1) into partitions based on the median value. Here, the processor 14 partitions the initial partition 44 in the compound pool 40(1) into a first partition 46(1) and a second partition 46(2) to form a second compound pool 40(2) shown in FIG. 6 based on the median value for the selected descriptor determined at step 140. The vertical axis M(1) in FIG. 6 depicts the median value.

Basically, the processor 14 determines whether the value of the selected descriptor for each database compound 42 of the initial partition 44 in the compound pool 40(1) is above or below the median value. If a database compound 42 has a value for the selected descriptor that is above the median value, the processor 14 assigns a value of “1” to the compound 42, although other types of identifiers may be used. On the other hand, if a database compound 42 has a descriptor value that is below the median value then the processor 14 assigns a value of “0” to the compound 42, although again, other types of identifiers may be used. Here, the processor 14 associates database compounds 42 that are assigned a value of “0” (i.e., below the median) to the first partition 46(1) and associates database compounds 42 that are assigned a value of “1” (i.e., above the median) to the second partition 46(2). Additionally, each of the database compounds 42 are assigned a unique bit string or partition code based on which of the first partition 46(1) and the second partition 46(2) the compounds 42 are associated with. The bit string is a unique signature that is used by the processor 14 to identify the partition that the compounds 42 belong to.

At step 155, the processor 14 executes the instructions stored in the memory 18(1) which comprise the descriptor system 20 to determine whether any of the descriptors determined to be suitable for calculating useful median values at step 120 remain. If only one descriptor was determined to be suitable for calculating useful median values, or if a plurality of descriptors were determined to be suitable, but only one descriptor was desired to be used, then no descriptors remain and the NO branch is followed. If several descriptors were determined to be suitable for calculating useful median values (and several descriptors were desired to be used), and there are suitable descriptors remaining that have not been used as described in connection with steps 130-150, then the YES branch is followed. It should be noted that each time the YES branch is followed, steps 130-150 are performed using suitable descriptors that have not been used before as described in connection with steps 130-150.

Here, the user of the computer 12 arbitrarily decided to use just two of the descriptors determined to be suitable as explained above in connection with step 120. As described above, the first descriptor was used to create the first partition 46(1) and the second partition 46(2) in the second compound pool 40(2) shown in FIG. 6. Therefore, the YES branch is followed and steps 130-150 are performed in the same manner described above, except the second descriptor determined to be suitable at step 120 is used instead of the first descriptor and the second compound pool 40(2) is used instead of the first compound pool 40(1). As a result, at step 150, the processor 14 partitions the first partition 46(1) in the compound pool 40(2) into a first sub-partition 48(1) and a second sub-partition 48(2), and the second partition 46(2) in the compound pool 40(2) into a third sub-partition 48(3) and a fourth sub-partition 48(4) to form a third compound pool 40(3) shown in FIG. 7 based on the median value of the second descriptor. Again, the vertical axis M(1) depicts the median value. Also, the horizontal axis M(2) in

FIG. 7 depicts the median value for the second descriptor in each of the partitions. At step 155, since the second suitable descriptor was used, no descriptors remain and the NO branch is followed.

At step 160, the processor 14 executes the instructions stored in the memory 18(1) which comprise the partition selection system 28 to select one or more of the database compounds 42 from each of the first sub-partition 48(1), the second sub-partition 48(2), the third sub-partition 48(3) and the fourth sub-partition 48(4) to form subgroups of database compounds 42 representative of all the compounds in each sub-partition 48(1)-48(4). The computer 12 sends the one or more selected database compounds 42 to the display device 30, where the compounds 42 or information describing the compounds is displayed and the method ends.

Another aspect of the system for identifying a small subgroup of compounds representative of a larger set of compounds includes the partition selection system determining a partition median value for each of the descriptor values for the compounds within a partition and selecting from the partition one or more compounds that have each descriptor value being within a predetermined range of values away from a corresponding partition median value to represent the compounds within the partition.

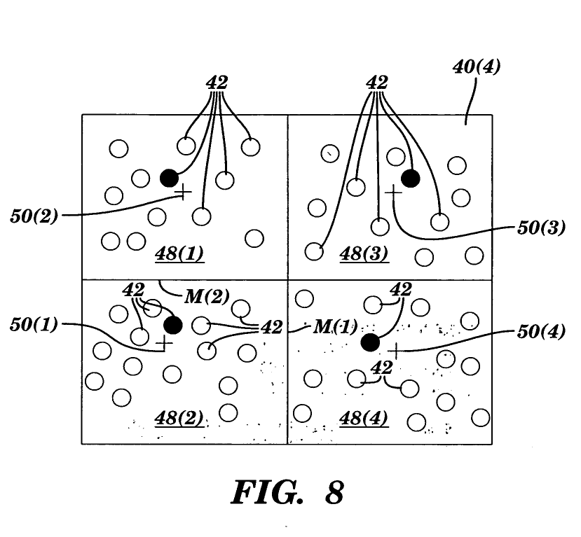

Steps 100-160 are performed in the same manner described above, except step 160 is performed as described herein. In this embodiment, the compound pool 40(4) illustrated in FIG. 8 is identical to the compound pool 40(3) illustrated in FIG. 7, except as described herein. Referring to FIG. 8, the processor 14 executes the instructions stored in the memory 18(1) which comprise the partition selection system 28 to determine quartile values 50(1)-50(4) for each of the sub-partitions 48(1)-48(4), respectively. Each of the quartile values 50(1)-50(4) represents the intersection point of the median values of each descriptor value for each of the database compounds 42 that were used to form each sub-partition. The processor 14 selects a compound, depicted as the compound 42 shown as a filled circle in FIG. 8, from each of the sub-partitions 48(1)-48(4) based on the compound (i.e., filled database compound 42) having the closest scaled Euclidian distance from the quartile values 50(1)-50(4) (Meier et al., “Statistical Methods in Analytical Chemistry,” John Wiley & Sons, New York (2000), which is hereby incorporated by reference herein in its entirety). Further, the processor 14 scales the Euclidian distances by dividing the distance by the range of each descriptor value. This procedure essentially selects compounds from the center of each of the sub-partitions 48(1)-48(4), thus avoiding boundary effects. In addition to quartile selections from each multiply populated partition, singletons (i.e., any sub-partitions containing only one compound, none of which are shown in this example) are included.

### EXAMPLE 1

An example of the operation of the system 10 is provided below. In this example, the system 10 and the steps 100-160 are performed to accomplish the identification of a small subgroup of compounds representative of a larger set of compounds. Further, the system 10 and the steps 100-160 are the same as described above, except as described herein. In this particular example, the compound database 24, and hence the compound pool 40(1), comprises about 300,000 compounds from the Available Chemicals Directory (“ACD”) (Available Chemicals Directory, MDL Information Systems, Inc., 14600 Catalina Street, San Leandro, Calif. 94577) (a portion of which is illustrated in FIG. 4), although other sources for the database compounds 42 may be used.

In this example, the descriptor database 25 includes a total of 147 1D, 2D and implicit 3D descriptors (Xue et al., “Accurate Partitioning of Compounds Belonging to Diverse Activity Classes,” J. Chem. Inf. Comput. Sci. 42:757-764 (2002), which is hereby incorporated by reference in its entirety) and a publicly available set of 166 structural keys (MACCS keys, MDL Information Systems, Inc., 14600 Catalina Street, San Leandro, Calif. 94577, which is hereby incorporated by reference in its entirety). Implicit 3D descriptors refer to a class of composite descriptors that map diverse properties to molecular surfaces approximated from 2D representations of molecules (Labute, “A Widely Applicable Set of Descriptors,” J. Mol. Graph. Model. 18:464-477 (2000), which is hereby incorporated by reference in its entirety).

In this example, the processor 14 executes the instructions stored in the memory 18(1) which comprise the descriptor system 20 to remove “exotic” database compounds 42 that would distort the descriptor values distributions. To accomplish this, the processor 14 calculates median absolute deviations (Meier et al., “Statistical methods in analytical chemistry,” John Wiley & Sons, New York (2000), which is hereby incorporated by reference in its entirety), defined as Mad =|x—M|/D, where “x” stands for each descriptor value in a population, “M” is the median value of the population of database compounds 42, and “D” is the median of |x—M|. Mad values essentially correspond to standard deviations but do not depend on the presence of normal data distributions. In this example, database compounds 42 were omitted from the compound database 24 if their Mad values were greater than nine for at least 10 of the selected descriptors. This stringent protocol was applied to remove only those database compounds 42 whose presence would skew distributions to a degree that the compound 42 would be separated from all others.

The processor 14 executes the instructions stored in the memory 18(1) which comprise the descriptor system 20 to utilize the Shannon entropy (“SE”) for descriptor analysis (Shannon et al., “The Mathematical Theory of Communication,” University of Illinois Press, Urbana (1963); Godden et al., “Variability of Molecular Descriptors in Compound Databases Revealed by Shannon Entropy Calculations,” J. Chem. Inf. Comput. Sci., 40:796-800 (2000); Godden et al., “Chemical Descriptors With Distinct Levels of Information Content and Varying Sensitivity to Differences Between Selected Compound Databases Identified by SE-DSE Analysis,” J. Chem. Inf. Comput. Sci. 42:87-93 (2002), which are hereby incorporated by reference in their entirety).

Further, the processor 14 executes the instructions stored in the memory 18(1) which comprise descriptor system 20 to select descriptors with detectable and significant information content (Godden et al., “Chemical Descriptors With Distinct Levels of Information Content and Varying Sensitivity to Differences Between Selected Compound Databases Identified by SE-DSE Analysis,” J. Chem. Inf. Comput. Sci. 42:87-93 (2002), which is hereby incorporated by reference in its entirety). Thus, the Shannon entropy is defined as

SE=−Σpᵢ log₂ pᵢ

In this formulation, p is the sample probability of a data point to fall as a count c within a specific data range i, and p is obtained as

pᵢ=cᵢ/Σcᵢ

The logarithm to the base two is a scale factor which makes it possible to consider SE as a metric of information content. It can be rationalized as a binary detector of counts (i.e., does the count appear in a given data interval?). Histograms provide a convenient way to establish the bit framework for data representation (here, descriptor value distributions). The major advantage of this concept is that the information content of descriptors having very different distributions and value ranges can be compared. Since SE values calculated from histograms are bin number-dependent, descriptor variability may vary from zero for a single valued descriptor to a maximum of the logarithm to the base two of the number of chosen histogram bins. Therefore, it is useful to establish a bin-independent SE value, called a scaled SE, which can be directly compared, regardless of the number of histogram bins.

Scaled SE values are calculated from histograms (Godden et al., “Variability of Molecular Descriptors in Compound Databases Revealed by Shannon Entropy Calculations,” J. Chem. Inf. Comput. Sci. 40:796-800 (2000); Godden et al., “Chemical Descriptors with Distinct Levels of Information Content and Varying Sensitivity to Differences Between Selected Compound Databases Identified by SE-DSE Analysis,” J. Chem. Inf. Comput. Sci., 42: 87-93 (2002), which are hereby incorporated by reference in their entirety). A scaled SE value is obtained by dividing an observed SE value by the maximum possible SE value for the number of bins used:

sSE=SE/log₂ (bins)

Based on the analysis of value distributions of many molecular descriptors in large compound collections (Godden et al., “Chemical Descriptors With Distinct Levels of Information Content and Varying Sensitivity to Differences Between Selected Compound Databases Identified by SE-DSE Analysis,” J. Chem. Inf. Comput. Sci. 42:87-93 (2002), which is hereby incorporated by reference in its entirety), generally applicable threshold values for low (e.g., <0.30), medium (e.g., 0.30-0.60), and high scaled SE (e.g., >0.6) have been established. From an original pool of 143 1D and 2D molecular property descriptors (Godden et al., “Chemical Descriptors With Distinct Levels of Information Content and Varying Sensitivity to Differences Between Selected Compound Databases Identified by SE-DSE Analysis,” J. Chem. Inf. Comput. Sci. 42:87-93 (2002), which is hereby incorporated by reference in its entirety), for example, descriptors having single values (and thus no information content) in the compound collections under investigation were excluded, yielding a total of 111 descriptors. Among these descriptors, scaled SE values ranged from 0.02 to 0.90. In addition, selected descriptors should display as little correlation as possible, as explained above.

Using correlated descriptors causes the data distributions to be skewed along the diagonal of correlation creating both empty and overpopulated partitions. To identify information-rich descriptors with little correlation, all n-by-n descriptor correlation coefficients were calculated for a set of 111 molecular property descriptors. This analysis revealed that it was improbable to identify combinations of completely uncorrelated chemical descriptors within the descriptor pool in the descriptor database 25 used in this example (Xue et al., “Molecular Descriptors for Effective Classification of Biologically Active Compounds Based on Principal Component Analysis Identified by a Genetic Algorithm,” J. Chem. Inf Compu. Sci. 40:801-809 (2000), which is hereby incorporated by reference in its entirety). Thus, the processor 14 executes the instructions stored in the memory 18(1) which comprise the genetic algorithm system 32 to optimize the descriptor combinations and minimizes correlation effects as much as possible.

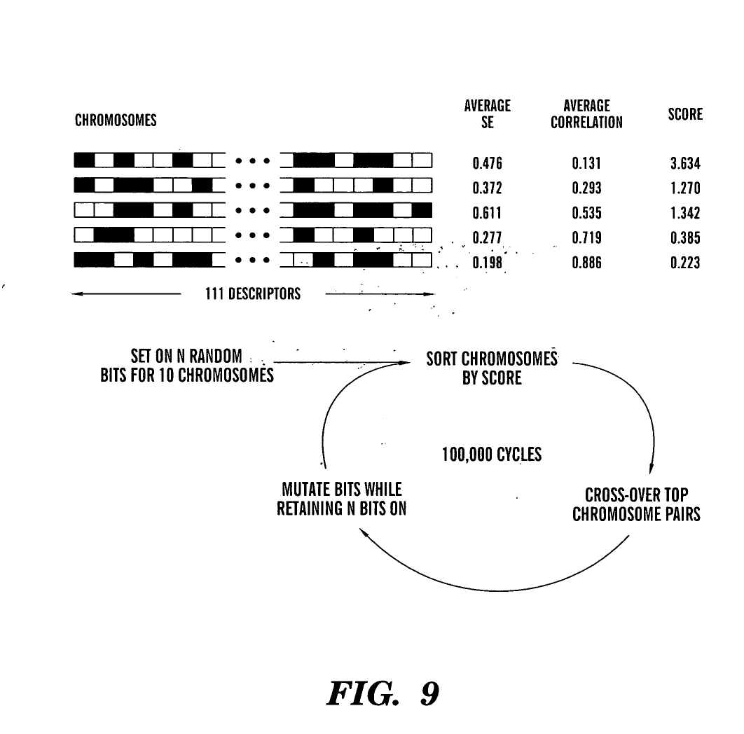

Referring to FIG. 9, a functional flow chart that depicts the operation of the processor 14 during execution of the instructions stored in the memory 18(1) which comprise the genetic algorithm system 32 in this example is shown. A set of chromosome representations stored in the memory 18(1) is run through a series or cycles of simulations during the execution of the instructions stored in the memory 18(1) which comprise the genetic algorithm system 32. The chromosome representations comprise randomly chosen descriptor combinations that are encoded in the chromosomes. Each of the chromosomes comprise 111 bits where each bit represents one of the descriptors. If a bit is set on (e.g., a value of “1”), the genetic algorithm system 32 adds the associated descriptor to the calculation. Further, the processor utilizes the scoring function S=<SE>/<CC>, where “CC” means correlation coefficient, to maximize average scaled SE values of the descriptor combinations and to minimize their average correlation coefficient. At each cycle, the crossover operation was applied to the top two chromosome pairs, the resulting chromosomes were mutated at a rate of 25%, and the calculations proceeded for 100,000 GA cycles.

In this example, the processor 14 executes the instructions stored in the memory 18(1) which comprise the descriptor system 20 to select sixteen descriptors which yield a total of 2¹⁶ or 65,536 possible partitions. The most favorable (i.e., information-rich and least correlated) descriptor combinations identified by the processor 14 by executing the instructions stored in the memory 18(1) which comprise the descriptor system 20 and the genetic algorithm system 32 in this example is reported in Table 1 below:

TABLE 1

| Descriptor | Scaled SE | Definition |
|---|---|---|
| Fcharge | 0.17 | sum of formal charges |
| PEOE_RPC- | 0.84 | relative negative partial charge |
| PEOE_VSA_FNEG | 0.86 | fractional negative vdw surface |
| | | area |
| PEOE_VSA_POL | 0.48 | total polar vdw surface area |
| a_aro | 0.48 | number of aromatic atoms |
| a_don | 0.28 | number of h-bond donor atoms |
| a_nP | 0.02 | number of phosphorous atoms |
| a_nS | 0.17 | number of sulfur atoms |
| b_rotR | 0.84 | fraction of rotatable bonds |
| b_triple | 0.06 | number of triple bonds |
| density | 0.56 | mass density |
| logP(o/w) | 0.49 | log octanol/water partition |
| | | coefficient |
| vsa_acc | 0.47 | vdw acceptor surface area |
| vsa_acid | 0.13 | vdw acidic surface area |
| vsa_don | 0.21 | vdw donor surface area |
| weimerPol | 0.61 | weiner polarity number |

The selected descriptors include various charge terms and approximate van der Waals surface area descriptors (Labute, P., “A widely applicable set of descriptors,” J. Mol. Graph. Model, 18: 464-477 (2000), which is hereby incorporated by reference herein in its entirety), as well as atom or bond counts and some bulk properties. The descriptor combination set forth in Table 1 above has an average SE value of 0.42 and an average absolute value of the pairwise correlation coefficient of 0.14.

Initially, salts and noncovalent complexes were removed from the compound database 24 (i.e., ACD) in this example, yielding a total of 231,187 compounds. The processor 14 executes the instructions stored in the memory 18(1) which comprise descriptor system 20 to perform Mad calculations on the database compounds 42 using the 111 descriptors to remove unusual or exotic compounds, as described above. These calculations further reduced the number of database compounds 42 to 225,929 database compounds 42. Of the 65,536 theoretically possible partitions, a total of 8,103 populated partitions are produced in this example, thus yielding an occupancy rate of 12.4%.

This illustrates the cumulative effects of descriptor correlations, even if they are relatively small. The obtained ACD partitions are variably populated and include 1,191 singletons. The largest partition in this example includes a total of 1,918 ACD database compounds 42. Filtering of the database compounds 42 revealed that 16% of the selected compounds had undesired reactive groups (Hann et al., “Strategic pooling of compounds for high-throughput screening,” J. Chem. Inf. Comput. Sci., 39: 897-902 (1999), which is hereby incorporated by reference herein in its entirety), and that 79% had between one and seven desired pharmacophore groups (Muegge et al., “Simple Selection Criteria for Drug-Like Chemical Matter,” J. Med. Chem., 44: 1841-1846 (2001), which is hereby incorporated by reference herein in its entirety), and 87% followed Lipinski's rules (Lipinski et al., “Experimental and Computational Approaches to Estimate Solubility and Permeability in Drug Discovery and Development Settings,” Adv. Drug. Deliv. Rev., 23:3-25 (1997), ), which is hereby incorporated by reference herein in its entirety). These relatively favorable characteristics were in part due to the fact that several thousand unusual compounds were removed from the ACD by Mad analysis prior to partitioning as described above.

The processor 14 executes the instructions stored in the memory 18(1) which comprise the partition selection system 28 in this example to select a representative subset of database compounds 42 from partitions based on the closest scaled Euclidian distance from the quartile (Meier et al., “Statistical methods in analytical chemistry,” John Wiley & Sons, New York (2000), which is hereby incorporated by reference in its entirety), an example of which is illustrated in FIG. 8. In addition to quartile selections from each multiply populated partition, all singletons (i.e., partitions containing only one compound) were included in the subset.

### EXAMPLE 2

Another example of the operation of the system 10 is provided below. In this example, the system 10 and the steps 100-160 are performed to accomplish library design. Further, the system 10 and the steps 100-160 are the same as described above, except as described herein. In this particular example, the compound database, and hence the compound pool 40(1), comprises a pool of approximately 2.5 million compounds collected from catalogs of various chemistry vendors. Further, in this example, the target library size is about 100,000 database compounds 42 in each partition. Thus, a total of 19 descriptors were selected for partitioning for this example.

The descriptor set in this example has an average absolute value of the correlation coefficient of 0.13. In these calculations, a partition occupancy rate of 21% was achieved and a total of 110,039 compounds were selected. In this more medicinal chemistry-oriented library, only 2% of the compounds had undesired reactive groups, 92% had between one and seven desired pharmacophore groups, and 83% were within the “Lipinski rule-of-5.” Selection of this library from a large source revealed the computational efficiency and potential of the system 10 for library design. Excluding initial calculations of descriptor values for the database compounds 42, which had already been completed for other purposes (Godden et al., “Chemical Descriptors with Distinct Levels of Information Content and Varying Sensitivity to Differences Between Selected Compound Databases Identified by SE-DSE Analysis,” J. Chem. Inf. Comput. Sci., 42: 87-93 (2002), which is hereby incorporated by reference herein in its entirety), median value statistics, partitioning and code assignments only required approximately two hours on a computer 12 where the processor 14 comprises a 14,600 MHz PC processor.

### EXAMPLE 3

Another example of the operation of the system 10 is provided below. In this example, the system 10 performs steps 100-160 to accomplish the classification of biologically active compounds. Further, the system 10 and the steps 100-160 are the same as described above, except as described herein. In this particular example, the compound database 24, and hence the compound pool 40(1), comprises 317 compounds belonging to 21 different biological activity classes (Xue et al., “Accurate Partitioning of Compounds Belonging to Diverse Activity Classes,” J. Chem. Inf. Comput. Sci., 42:757-764 (2002)), which is hereby incorporated by reference in its entirety), including diverse sets of enzyme inhibitors, receptor agonists and antagonists, and both synthetic and naturally occurring compounds.

The composition of the compound database 24 in this example is summarized below in Table 2:

TABLE 2
Biological Activity Classes

| Biological activity | No. of compds |
|---|---|
| Cyclooxygenase-2 (Cox-2) inhibitors | 17 |
| Tyrosine kinase (TK) inhibitors | 20 |
| HIV protease inhibitors | 18 |
| H3 antagonists | 21 |
| Benzodiazepine receptor ligands | 22 |
| Serotonin receptor ligands (5-HT) | 21 |
| Carbonic anhydrase II inhibitors | 22 |
| β-lactamase inhibitors | 14 |
| Protein kinase C inhibitors | 15 |
| Estrogen antagonists | 11 |
| Antihypertensive (ACE inhibitor) | 17 |
| Antiadrenergic (β-receptor) | 16 |
| Glucocorticoid analogues | 14 |
| Angiotensin ATI antagonists | 10 |
| Aromatase inhibitors | 10 |
| DNA topolsomerase I inhibitors | 10 |
| Dinhydrofolate reductase inhibitors | 11 |
| Factor Xa inhibitors | 14 |
| Farnesyl transferase inhibitors | 10 |
| Matrix metalloproteinase inhibitors | 12 |
| Vitamin D analogues | 12 |

In addition, 2,000 randomly collected background compounds from the ACD (Available Chemicals Directory, MDL Information Systems, Inc., 14600 Catalina Street, San Leandro, Calif. 94577, which is hereby incorporated herein by reference in its entirety) were added to the compound database 24 to further increase the degree of difficulty for compound classification for this example.

In this example, the descriptor database 25 includes a total of 147 1D, 2D and implicit 3D descriptors (Xue et al., “Accurate Partitioning of Compounds Belonging to Diverse Activity Classes,” J. Chem. Inf. Comput. Sci. 42:757-764 (2002), which is hereby incorporated by reference in its entirety) and a publicly available set of 166 structural keys (MACCS keys, MDL Information Systems, Inc., 14600 Catalina Street, San Leandro, Calif. 94577, which is hereby incorporated by reference in its entirety). Implicit 3D descriptors refer to a class of composite descriptors that map diverse properties to molecular surfaces approximated from 2D representations of molecules (Labute, “A Widely Applicable Set of Descriptors,” J. Mol. Graph. Model. 18:464-477 (2000), which is hereby incorporated by reference in its entirety). In this example, however, the descriptors stored in the descriptor database 25 may correlate with each other without hindering performance.

The processor 14 executes the instructions stored in the memory 18(1) which comprise the descriptor system 20 and the MOE system 34 to calculate values for all of the descriptors stored in the descriptor database 25. Nevertheless, those descriptors that occurred in the best scoring combinations, as identified by the processor 14 executing the instructions stored in the memory 18(1) which comprise the genetic algorithm system 32, are also defined below in Table 3:

TABLE 3
Definitions of Selected Descriptors

| | | median | Median |
|---|---|---|---|
| descriptor | definition | (317) | (2317) |
| apol | sum of the atomic | 55.26 | 44.49 |
| | polarizabilitics of all atoms | | |
| a_aro | number of aromatic atoms | 12 | 10 |
| a_don | number of H-bond donors | 2 | 1 |
| a_heavy | number of heavy atoms | 26 | 21 |
| a_hyd | number of hydrophobic | 17 | 14 |
| | atoms | | |
| a_nN | number of nitrogen atoms | 3 | 1 |
| a_nF | number of fluorine atoms | 0 | 0 |
| a_nS | number of sulfur atoms | 0 | 0 |
| a_nI | number of iodine atoms | 0 | 0 |
| b_heavy | number of bonds between | 29 | 22 |
| | heavy atoms | | |
| b_ar | number of aromatic bonds | 12 | 11 |
| b_double | number of double | 1 | 1 |
| | nonaromatic bonds | | |
| chi0 | atomic connectivity index | 19.07 | 15.28 |
| | (order 0)²³ | | |
| chilv_C | carbon valence | 5.93 | 4.55 |
| | connectivity index (order | | |
| | 1) | | |
| chil_C | carbon connectivity index | 7.83 | 6.02 |
| | (order 1) | | |
| diameter | largest value in the | 13 | 11 |
| | distance matrix²⁴ | | |
| KicrA3 | third kappa shape index²³ | 3.87 | 3.59 |
| PEOE_RPC | relative negative partial | 0.17 | 0.21 |
| | charge²⁵ | | |
| PEOE_VSA + 3 | sum of v₁ where p₁ is in the | 10.68 | 0.00 |
| | range [0.15, 0.20] | | |
| PEOE_VSA − 1 | sum of v₁ where p₁ is in the | 55.88 | 56.24 |
| | range [−0.10, −0.05] | | |
| PEOE_VSA − 3 | sum of v₁ where p₁ is in the | 0.00 | 0.00 |
| | range [−0.20, −0.15] | | |
| PEOE_VSA − 4 | sum of v₁ where p₁ is in the | 5.51 | 0.00 |
| | range [−0.25, −0.20] | | |
| PEOE_VSA − 5 | sum of v₁ where p₁ is in the | 13.57 | 13.57 |
| | range [−0.30, −0.25] | | |
| PEOE_VSA_POS | total positive van der | 195.83 | 146.89 |
| | Waals surface area | | |
| PEOE_VSA_FPNEG | fractional negative polar | 0.09 | 0.08 |
| | van der Waals surface area | | |
| PEO_VSA_FHYD | fractional hydrophobic van | 0.84 | 0.86 |
| | der Waals surface area | | |
| SlogP_VSA2 | sum of v₁ such that L₁ is in | 23.86 | 19.41 |
| | (−0.2, 0] | | |
| SlogP_VSA7 | sum of v₁ such that L₁ is in | 124.85 | 88.22 |
| | (0.25, 0.30] | | |
| SMR_VSA0 | sum of v₁ such that R₁ is in | 32.16 | 23.86 |
| | [0.0.11] | | |
| SMR_VSA1 | sum of v₁ such that R₁ is in | 36.39 | 22.00 |
| | (0.11, 0.26] | | |
| SMR_VSA4 | sum of v₁ such that R₁ is in | 6.37 | 2.76 |
| | (0.39, 0.44] | | |
| SMR_VSA5 | sum of v₁ such that R₁ is in | 158.79 | 126.75 |
| | (0.44, 0.485] | | |
| VAdjMa | vertex adjacency | 5.86 | 5.46 |
| | information (magnitude) | | |
| VDistEq | vertex distance equality | 3.44 | 3.24 |
| | index | | |
| VDistMa | vertex distance magnitude | 9.13 | 8.47 |
| | index | | |
| vsa_acc | VDW surface area of | 27.93 | 19.25 |
| | hydrogen-bond acceptors | | |
| vsa_don | VDW surface area of | 0.00 | 0.00 |
| | hydrogen-bond donors | | |
| vsa_other | VDW surface area of | 35.78 | 27.10 |
| | nondonor/-acceptor atoms | | |
| vsa_pol | VDW surface area of polar | 19.25 | 0.00 |
| | atoms | | |
| vdw_vol | VDW volume calculated | 480.21 | 389.72 |
| | using a connection table | | |
| Zagreb | Zagreb index | 142 | 106 |

In Table 3 above: v₁ is the van der Waals (VDW) surface area of atom i; pi represents the partial charge of atom i calculated using a PEOE method (Gasteiger et al., “Iterative Partial Equalization or Orbital Electronegativity—A Rapid Access to Atomic Charges,” Tetrahedron, 36: 3219-3228 (1980), which is hereby incorporated by reference herein in its entirety); Lᵢ denotes the contribution to logP(o/w) for atom i as calculated in the SlogP descriptor (Wildman et al., “Prediction of Phsiochemical Parameters by Atomic Contributions,” J. Chem. Inf. Comput. Sci., 39: 868-873 (1999), which is hereby incorporated by reference herein in its entirety); and Rᵢ denotes the contribution to molar refractivity for atom i as calculated in the SMR descriptor (Wildman et al., “Prediction of Phsiochemical Parameters by Atomic Contributions,” J. Chem. Inf. Comput. Sci. 39: 868-873 (1999), which is hereby incorporated by reference herein in its entirety). The design of “VSA” descriptors has also been reported (Labute, “A Widely Applicable Set of Descriptors,” J. Mol. Graph. Model. 18:464-477 (2000), which is hereby incorporated by reference herein in its entirety). For each listed descriptor in Table 3 above, calculated median values are shown for both compound databases analyzed here (consisting of 317 and 2,317 molecules, respectively).

Since the system 10 relies on the calculation of medians of descriptor value distributions, binary or two-state descriptors, such as structural fragments, are not applied here. The only requirement for the preselection of property descriptors for system 10 is that they have nonzero descriptor entropy for which meaningful median values can be calculated (Godden et al., “Variability of Molecular Descriptors in Compound Databases Revealed by Shannon Entropy Calculations,” J. Chem. Inf. Comput. Sci. 40:796-800 (2000); Godden et al., “Chemical Descriptors With Distinct Levels of Information Content and Varying Sensitivity to Differences Between Selected Compound Databases Identified by SE-DSE Analysis,” J. Chem. Inf. Comput. Sci. 42:87-93 (2002), which are hereby incorporated by reference in their entirety). This effectively reduces the number of suitable property descriptors from 147 to 130.

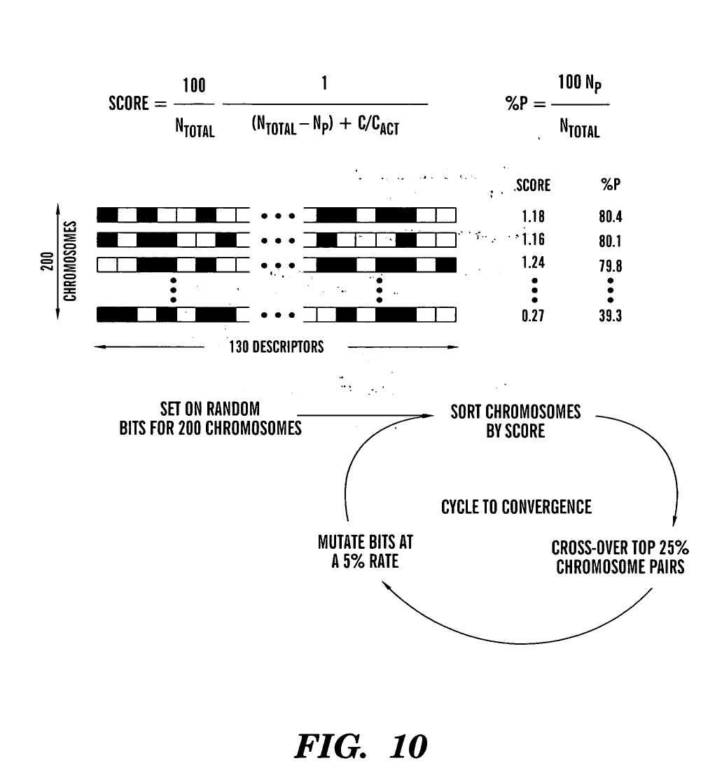

Referring to FIG. 10, a functional flow chart that depicts the operation of the processor 14 during execution of the instructions stored in the memory 18(1) which comprise the genetic algorithm system 32 in this example is shown. A set of chromosome representations stored in the memory 18(1) is run through a series or cycles of simulations during the execution of the instructions which comprise the genetic algorithm system 32. The chromosome representations comprise randomly chosen descriptor combinations that are encoded in the chromosomes. The partitioning calculations are carried out and evaluated via a scoring function, which is then optimized by the processor 14 executing the instructions stored in the memory 18(1) which comprise the genetic algorithm system 32 during each cycle by altering descriptor combinations using mutation (inversion of single bit positions) and crossover (bit segment swapping) operations until a predefined convergence criterion is reached. Here, the design of chromosomes that are used by the processor 14 during execution of the instructions stored in the memory 18(1) which comprise the genetic algorithm system 32 in this example is simpler than the chromosomes used by other genetic algorithms, such as GA-PCA.

Here, initially assembled chromosomes only represent the total number of available descriptors, 130 in this case, and each bit, if set on, adds a specific descriptor to the calculations. The first 200 chromosomes were randomly generated with an initial occupancy rate of less than 10%, and the top scoring 25% of the chromosomes were subjected to pairwise crossover operations, followed by random mutation of all remaining chromosomes at a rate of 5%. The processor 14 continued the cycles of executing the instructions stored in the memory 18(1) which comprise the genetic algorithm system 32 until no change in score for 1000 cycles was observed by the processor 14.

In this example, two independent genetic algorithm system 32 optimizations were carried out: one for where the compound database 24 has just active compounds (317 molecules), and another where the database 24 has both the active compounds (317 molecules) and the background compounds (2,317 molecules). Where the compound database 24 has just the 317 molecules, convergence was reached after 3,502 cycles. Where the compound database 24 has 2,317 molecules, 13,657 cycles were required to reach convergence.

In this example, the general goal with regard to compound classification is to obtain as many compounds as possible in “pure” partitions or cells (that exclusively consist of molecules sharing the same activities), while minimizing the number of compounds in mixed partitions (i.e., consisting of molecules having different activity) or singletons (active molecules not predicted to be similar to others). Furthermore, the descriptor combinations that yield the best predictive performance should be identified.

The processor 14 executes the instructions stored in the memory 18(1) which comprise the genetic algorithm system 32 in this example to implement an appropriate scoring function and algorithm to facilitate descriptor selection. Therefore, the following scoring function is implemented and optimized by the processor 14 during cycles:

$$S = \frac{100}{{N}_{total}} \times \frac{1}{{N}_{total} - {N}_{p} ) + C / {C}_{act}}$$

In this formulation, Nₜₒₜₐₗ is the total number of active compounds (here 317), and Nₚ is the number of compounds occurring in pure partitions. Both the number of compounds in mixed classes and singletons are regarded as classification failures. In addition, C is the total number of partitions that contain active compounds (pure, mixed, or singletons) and C_{act} is the number of different activity classes in the database (21 in this case). Thus, the scoring function also attempts to minimize the total number of “active” partitions or cells that are created.

Consequently, high scores are obtained if many compounds occur in a small number of pure partitions. A scaling factor of 100 is applied to obtain top scores greater than 1. The addition of background compounds increases the degree of difficulty for the classification calculations because the statistical probability of producing mixed partitions or cells becomes significantly higher. In addition, as an intuitive measure of overall classification accuracy for each calculation, we also define the fraction of compounds in pure partitions as % P=100·Nₚ/Nₜₒₜₐₗ. This additional metric is not applied to guide descriptor selection during GA cycles but is constantly monitored by the processor 14.

The present invention also relates to a system for virtual compound screening that includes a bait compound system, a descriptor system, a median determination system, a partitioning system, a partition recombination system, and a selection system. The bait compound system combines information representing a plurality of unidentified compounds with information representing a plurality of bait compounds having known biological activities to form a set of compounds. The descriptor system obtains one or more descriptor values for each of the unidentified compounds and for each of the bait compounds in the set of compounds, and the median determination system determines a median value for each of the descriptor values for the set of compounds. The partitioning system partitions the set of compounds into a plurality of partitions based on each median value, and the partition recombination system then recombines partitions which have at least two bait compounds to form a recombined set of compounds. A selection system then selects the recombined set of compounds for analysis of biological activity if an approximate target number of unidentified compounds remain in the recombined set of compounds.

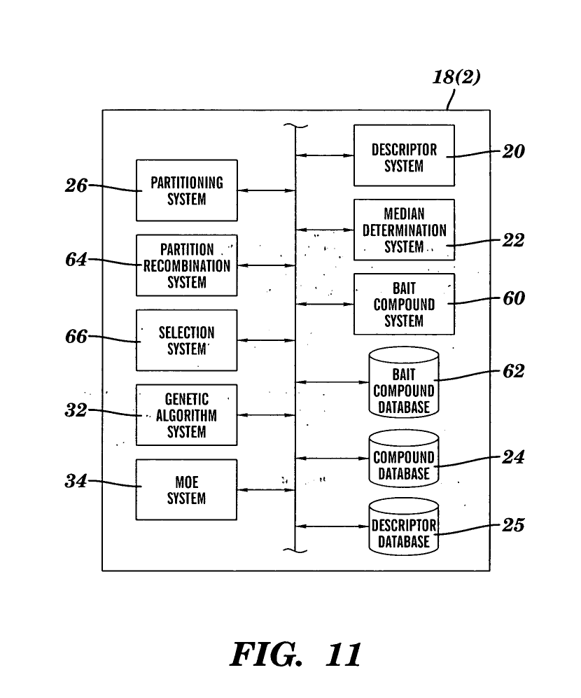

In this embodiment of the present invention, like reference numbers in FIGS. 11-17 are identical to those in and described with reference to FIGS. 1-10. Also, the system 10 in this embodiment is identical to the system 10 in other embodiments, except here the system 10 includes memory 18(2), shown in FIG. 11, substituted for memory 18(1). Further, memory 18(2) is the same as the memory 18(1), but also includes a bait compound system 60, a bait compound database 62, a partition recombination system 64 and a selection system 66, and does not include a partition selection system 28.

In this embodiment, the compound database 24 comprises data representing about 1.34 million compounds collected from various compound sources and vendor catalogs that are organized in the memory 18(2).

The bait compound system 60 comprises instructions stored in the memory 18(2) which when executed by the processor 14 accesses the bait compound database 62 and the compound database 24, and introduces a plurality of bait compounds from the bait compound database 62 into a pool of unknown compounds from the compound database 24 during operation of the system 10 during each recursion as explained in greater detail herein below.

The bait compound database 62 comprises data representing a plurality of randomly selected compounds obtained from a structurally diverse biological activity database (Xue et al., “Accurate Partitioning of Compounds Belonging to Diverse Activity Classes,” J. Chem. Inf. Comput. Sci. 42:757-764 (2002), which is hereby incorporated by reference herein in its entirety), which are organized in the memory 18(2). Further, the compounds in the bait compound database 62 represent different classes of molecules with specific biological activity. Examples of bait compounds 72 comprise benzodiazepine receptor ligands, serotonin receptor ligands, tyrosine kinase inhibitors, histamine H3 antagonists, cyclooxygenase-2 inhibitors, HIV protease inhibitors, carbonic anhydrase II inhibitors, β-lactamase inhibitors, protein kinase C inhibitors, estrogen antagonists, antihypertensive (ACE inhibitor), antiadrenergic (β-receptor), glucocorticoid analogues, angiotensin AT1 antagonists, aromatase inhibitors, DNA topoisomerase I inhibitors, dihydrofolate reductase inhibitors, factor Xa inhibitors, farnesyl transferase inhibitors, matrix metalloproteinase inhibitors, and vitamin D analogues.

The partition recombination system 64 comprises instructions stored in the memory 18(2) which when executed by the processor 14 recombines compounds from the compound database 24 and bait compounds from the bait compound database 62 which are in one or more compound partitions that satisfy a “co-partitioning” rule, which will be described in greater detail further herein below, to form a recombined compound pool.

The selection system 66 comprises instructions stored in the memory 18(2) which when executed by the processor 14 determines whether the number of database compounds in a recombined compound pool (i.e., a compound pool formed by recombining compound partitions that satisfy the co-partitioning rule) is equal to, less than or greater than a target number of remaining compounds.

The present invention also relates to a method for virtual compound screening. The method will now be described in the context of being carried out by the system 10 with reference to FIGS. 11-17. Basically, the method includes combining a plurality of unknown compounds with a plurality of bait compounds having known biological activities to create a set of compounds. One or more descriptor values are obtained for each of the unidentified compounds and for each of the bait compounds in the set of compounds. A median value is obtained for each of the descriptor values for the set of compounds and the set of compounds are partitioned into a plurality of partitions based on each median value. Partitions which have at least two bait compounds are recombined to form a recombined set of compounds, and the recombined set of compounds is selected for analysis of biological activity if an approximate target number of unidentified components remain in the recombined set of compounds.

By way of example only, a user operating computer 12 desires performing virtual screening of the compounds in the compound database 24. The computer 12 performs steps 100-120 in the same manner described above, except as described herein.

At step 100, the processor 14 executes the instructions stored in the memory 18(2) which comprise the bait compound system 60 to access the compound database 24 and the bait compound database 62 for further processing as described herein below.

At step 110, the processor 14 executes the instructions stored in the memory 18(2) which comprise the descriptor system 20 and the MOE system 34 to calculate values of the molecular property descriptors organized in the descriptor database 25 for each of the compounds in the compound database 24 and the bait compound database 62.

At step 120, the processor 14 executes the instructions stored in the memory 18(2) which comprise the descriptor system 20 to evaluate the descriptors to determine the optimal set of descriptors to use for the compounds in the compound database 24. Again, as in other embodiments and examples, the processor 14 selects descriptors that will be suitable for calculating useful median values in that they have high information content (Godden et al., “Chemical Descriptors With Distinct Levels of Information Content and Varying Sensitivity to Differences Between Selected Compound Databases Identified by SE-DSE Analysis,” J. Chem. Inf. Comput. Sci. 42:87-93 (2002), which is hereby incorporated by reference in its entirety). In this example, broad distribution of diverse values favor the calculation of meaningful median values (Godden et al., “Classification of Biologically Active Compounds by Median Partitioning,” J. Chem. Inf. Comput. Sci, 42 (2002), which is hereby incorporated by reference in its entirety).

However, in this embodiment, the processor 14 selects information-rich descriptors regardless of whether they correlate with each other or not. Thus, the processor 14 selects a set of descriptors comprising 127 diverse 1D and 2D molecular descriptors (Xue et al., “Accurate Partitioning of Compounds Belonging to Diverse Activity Classes,” J. Chem. Inf. Comput. Sci. 42:757-764 (2002); Godden et al., “Median Partitioning: A Novel Method for the Selection of Representative Subsets from Large Compound Pools,” J. Chem. Inf. Comput. Sci. 42:885-893 (2002), which are hereby incorporated by reference herein in their entirety).

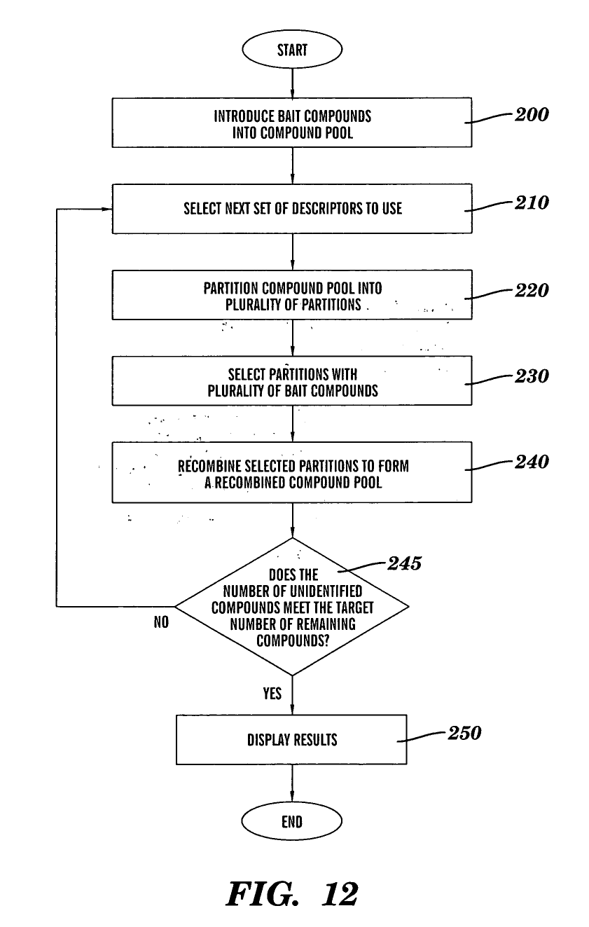

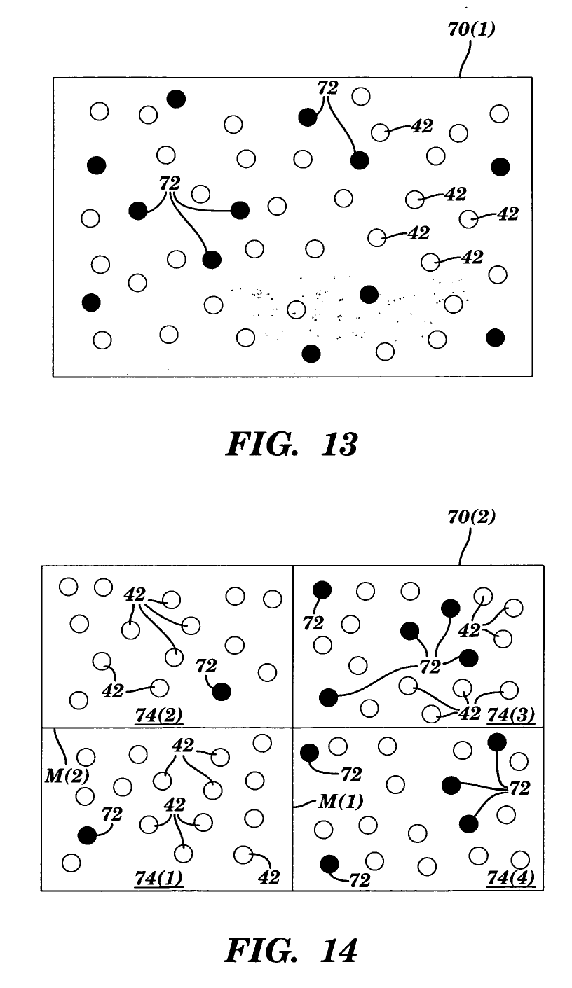

Referring to FIGS. 12-13 and beginning at step 200, the processor 14 executes the instructions stored in the memory 18(2) which comprise the bait compound system 60 to introduce a plurality of bait compounds 72 into a compound pool 70(1) having unknown database compounds 42 from the compound database 24. It should be noted that only a portion of the compounds from the bait compound database 62 and the compound database 24 are illustrated in FIGS. 13-17. Further, the reference numbers (e.g., 42 and 72) in FIGS. 13-17 are shown as identifying just some of the database compounds 42 and the bait compounds 72 in the compound pools 70(1)-70(2), 76(1)-76(2) and 80 for clarity, but it should be understood that all of the solid or filled circles in FIGS. 13-17 represent all of the bait compounds 72 and all of the transparent or unfilled circles represent all of the database compounds 42 obtained from the bait compound database 62 and compound database 24, respectively.

At step 210, the processor 14 executes the instructions stored in the memory 18(2) which comprise the descriptor system 20 to select the next set of one or more suitable descriptors. In this exemplary embodiment, each set of suitable descriptors comprise two suitable descriptors, although the set may comprise a fewer or greater number of descriptors. The processor 14 executes the instructions stored in the memory 18(2) which comprise the descriptor system 20 and the genetic algorithm system 32 to identify a set of suitable descriptors which will co-partition as many bait compounds 72 as possible. Referring back to FIG. 10, the processor 14 uses each of about 100 bits of a chromosome to determine whether a particular descriptor is included (i.e., if set on to “1”) or not (i.e., if set off to “0”) in the calculation of the associated fitness function. The processor 14 begins with 200 randomly generated chromosomes and the top scoring 40 (25%) are subjected to crossover and mutation operations (at a 5% mutation rate). The calculations are repeated until convergence is reached, in this case, 1,000 cycles without improving the score S.

The associated fitness function used by the processor 14 in this embodiment is defined as:

S=Act(cp)×Pa(pop),

where Act(p) is the total number of co-partitioned known active compounds and Pa(pop) is the total number of populated partitions. This fitness function directs the processor 14 to select descriptor sets that favor co-partitioning of known active compounds and, at the same time, maximally disperse the database molecules over unique partitions. This situation is thought to be optimal for obtaining a subset of database molecule most similar to the bait compounds.

Between twenty and thirty nine property descriptors are typically required to achieve the best observed level of performance based on the compound database 24 and bait compound database 62 used in this example, although a fewer or greater number of descriptors may be used. The distribution of descriptor categories is relatively similar for all compound classes. Prevalent is a descriptor type referred to herein as the surface property descriptors. These descriptors are designed to map various physical properties (e.g., partial atomic charges) to molecular surface segments approximated from 2D representations of molecules (Labute, “A Widely Applicable Set of Descriptors,” J. Mol. Graph. Model. 18:464-477 (2000), which is hereby incorporated by reference in its entirety) and have very high information content (Godden et al., “Chemical Descriptors With Distinct Levels of Information Content and Varying Sensitivity to Differences Between Selected Compound Databases Identified by SE-DSE Analysis,” J. Chem. Inf. Comput. Sci. 42:87-93 (2002), which is hereby incorporated by reference in its entirety).

At step 220, the compound pool 70(1) is partitioned into a first set of partitions 74(1)-74(4) to create a first partitioned pool 70(2), as shown in FIG. 14. Specifically, the processor 14 executes the instructions stored in the memory 18(2) which comprise the median determination system 22 to calculate the median value of the descriptors selected above at step 210 based on the descriptor values of the selected descriptor for all of the database compounds 42 and bait compounds 72 in the compound pool 70(1) that are calculated at step 110. The processor 14 then executes the instructions stored in the memory 18(2) which comprise the partitioning system 26 to partition the compound pool 70(1) into the first set of partitions 74(1)-74(4) based on the median values of the two suitable descriptors in this example. The vertical axis M(1) depicts the median value for the first descriptor, and the horizontal axis M(2) depicts the median value for the second descriptor. Additionally, each of the database compounds 42 and the bait compounds 72 in the first set of partitions 74(1)-74(4) is assigned a unique bit string based on which of the first set of partitions 74(1)-74(4) the compounds are from for identification purposes.

At step 230, the processor 14 executes the instructions stored in the memory 18(2) which comprise the partition recombination system 64 to examine the first set of partitions 74(1)-74(4) for determining which of the partitions has at least two bait compounds 72. As shown in FIG. 14, the first set of partitions 74(3) and 74(4) have at least two bait compounds 72 and partitions 74(1) and 74(2) have only one bait compound in each partition in this example. The processor 14 selects partitions with at least two bait compounds 72 to satisfy a “co-partitioning” rule, which means that only those partitions with two or more bait compounds 72 should be considered further. The rationale behind the co-partitioning rule is that having more bait compounds (e.g., at least two bait compounds 72) with known activities in a partition increases the probability that the unknown database compounds 42 in that same partition will have the same activities. Thus, the processor 14 selects the partitions 74(3) and 74(4) for this example.

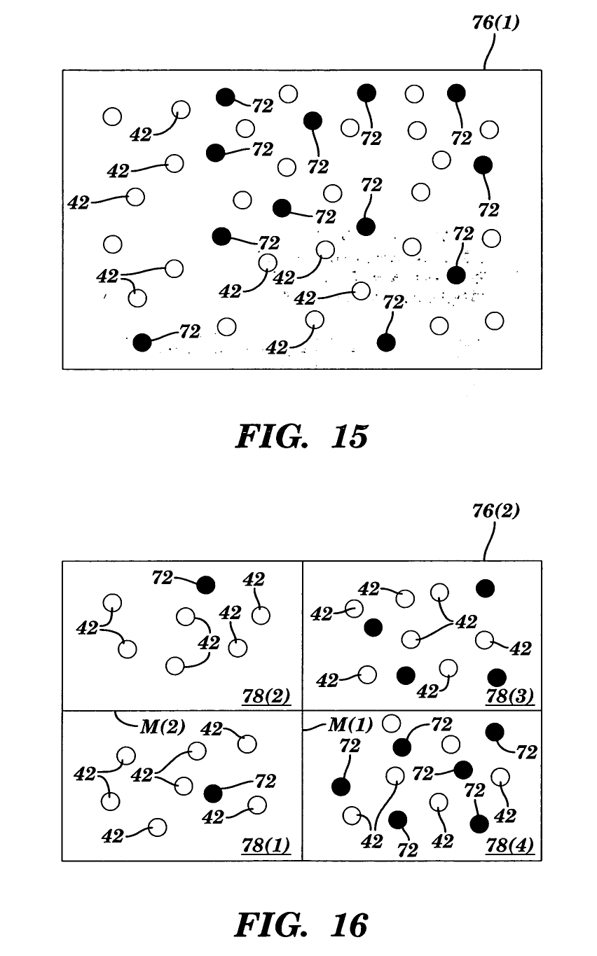

At step 240, the processor 14 executes the instructions stored in the memory 18(2) which comprise the partition recombination system 64 to recombine the database compounds 42 and the bait compounds 72 from the first set of partitions 74(3) and 74(4) into one pool to form the recombined pool 76(1) shown in FIG. 15. Further, the processor 14 reintroduces the bait compounds 72 from the first set of partitions 74(1) and 74(2) into the recombined pool 76(1). The database compounds 42 that are in the first set of partitions 74(1) and 74(2) are not considered further by the processor 14 in this example since the one bait compound 72 present in each of those partitions was not recognized as being similar to any other active compound (based on the descriptor values), thus violating the co-partitioning rule.

At step 245, the processor 14 executes the instructions stored in the memory 18(2) which comprise the selection system 66 to determine whether the number of database compounds 42 in the recombined pool 76(1) is equal to or lower than a target number. The target number (e.g., less than 100 compounds) is arbitrary and can be set at any time by the user of the computer 12. If the number of compounds 42 in the recombined pool 76(1) is equal to or less than the target number, then the YES branch is followed. If the number of compounds 42 remaining in the recombined pool 76(1) is greater than the target number, then the NO branch is followed and steps 200-245 are repeated (i.e., another “recursion”), except at step 210 a different set of suitable descriptors than any descriptors used previously is selected.

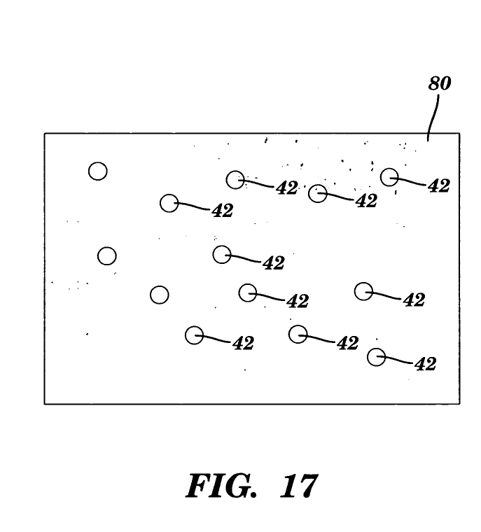

Here, the number of compounds 42 remaining in the recombined pool 76(1) is greater than the target number. As a result, the NO branch is followed and steps 210-245 are repeated as described herein. Thus, steps 210-220 are repeated to create a second set of partitions 78(1)-78(4) in a second partitioned compound pool 76(2), as shown in FIG. 16. Step 230 is repeated and the second set of partitions 78(3) and 78(4) are selected and recombined at step 240 to form the final compound pool 80 shown in FIG. 17 in this example. At step 245, the processor 14 determines that the number of compounds 42 in the final compound pool 80 is equal to or less than the target number and the YES branch is followed.

At step 250, the computer 12 sends the results, such as information describing the compounds 42 from the compound pool that was determined to have the number of remaining compounds 42 equal to or lower than a target number (e.g., final compound pool 80), to the display device 30. The display device 30 displays the results and the method ends.

### EXAMPLE 1

An example of the operation of the system 10 for performing virtual screening is provided below. In this example, the system 10 and the steps 100-120 and 200-250 are the same as described above, except as described herein. In this particular example, the system 10 operates to perform steps 100-120 and 200-250 as described above. An exemplary set of activity classes, a number of bait compounds 72 in each class, and the “hits” of unknown database compounds 42 per class in partitions resulting from the operation are shown below in Table 4:

TABLE 4

| | | Active database |
|---|---|---|
| Activity class | Baits compounds | molecules |
| Benzodiazepine | 10 | 49 |
| receptor ligands | | |
| Serotonin | 10 | 61 |
| receptor ligands | | |
| Tyrosine kinase | 10 | 25 |
| inhibitors | | |
| Histamine H3 | 10 | 42 |
| Antagonists | | |
| Cyclooxygenase- | 10 | 21 |
| 2 inhibitors | | |

Next, three independent analyses with five recursions (i.e., three separate operations of the system 10 with five recursions each) were carried out by the system 10 in this example and the results were averaged for each test case as shown below in Table 5:

TABLE 5

| | | | | | |
|---|---|---|---|---|---|
| | | | | | Im- |
| | | | Active | | prove- |
| Recursion | Database | Bait | database | Hit | ment |
| level | compounds | compounds | compounds | rate | factor |

Benzodiazepine receptor ligands

| | | | | | |
|---|---|---|---|---|---|
| 0 | 1340848 | 10 | 49 | 3.6e−05 | |
| 1 | 164423.7 | 8 | 35.7 | 0.00022 | 6.1 |
| 2 | 20596 | 7.7 | 24 | 0.0012 | 33.3 |
| 3 | 3268.7 | 7.3 | 15.7 | 0.0048 | 133.3 |
| 4 | 468.4 | 6.3 | 11.7 | 0.025 | 694.4 |
| 5 | 73.7 | 6.3 | 8.7 | 12% | 3333.3 |

Serotonin receptor ligands

| | | | | | |
|---|---|---|---|---|---|
| 0 | 1340860 | 10 | 61 | 4.6e−05 | |
| 1 | 172409.6 | 6 | 46.3 | 0.00027 | 5.9 |
| 2 | 19229 | 6.3 | 38 | 0.002 | 43.5 |
| 3 | 3366.7 | 5.7 | 28.7 | 0.0085 | 184.8 |
| 4 | 399.6 | 4 | 19.3 | 0.048 | 1043.5 |
| 5 | 62 | 4.3 | 13.3 | 21% | 4565.2 |

Tyrosine kinase inhibitors

| | | | | | |
|---|---|---|---|---|---|
| 0 | 1340824 | 10 | 25 | 1.9e−05 | |
| 1 | 205276 | 10 | 19 | 9.3e−05 | 4.9 |
| 2 | 24359.7 | 9.3 | 16 | 0.00066 | 34.7 |
| 3 | 3980.4 | 9.3 | 13.7 | 0.0034 | 178.9 |
| 4 | 480.3 | 8 | 12.3 | 0.026 | 1368.4 |
| 5 | 74.3 | 8 | 10 | 13% | 6842.1 |

Histamine H3 antagonists

| | | | | | |
|---|---|---|---|---|---|
| 0 | 1340841 | 10 | 42 | 3.1e−05 | |
| 1 | 274605.3 | 6.7 | 19 | 6.9e−05 | 2.2 |
| 2 | 29417.3 | 3 | 9.3 | 0.00032 | 10.3 |
| 3 | 3718.3 | 2.7 | 4.3 | 0.0012 | 38.7 |
| 4 | 536.6 | 2.3 | 3.3 | 0.0062 | 0.19 |
| 5 | 59.3 | 2 | 2 | 3.4% | 1096.8 |

Cyclooxygenase-2 inhibitors

| | | | | | |
|---|---|---|---|---|---|
| 0 | 1340820 | 10 | 21 | 1.6e−05 | |
| 1 | 191183.7 | 7.7 | 15.7 | 8.2e−05 | 5.1 |
| 2 | 21927 | 7 | 10 | 0.00046 | 28.8 |
| 3 | 2866.3 | 7.3 | 8 | 0.0028 | 175.0 |
| 4 | 467.6 | 5.3 | 4.3 | 0.0092 | 575.0 |
| 5 | 70 | 4 | 2.3 | 3.3% | 2062.5 |

In Table 5, the final results are shown in bold face at recursion level 5. Recursion level 0 shows the initial database composition. For each recursion, the total number of bait compounds that co-partition is reported. Also shown is the total number of active compounds found among the database compounds that fall into partitions containing at least two bait molecules. Hit rate is calculated by dividing the number of active molecules (excluding baits) by the total number of compounds in these partitions. For recursion level 0, hit rate reports the fraction of active molecules (excluding baits) in the database. Improvement factor over random compound selection is calculated by dividing the hit rate by the fraction of active molecules (recursion level 0).

Table 6 below shows the descriptor statistics for the final recursions:

TABLE 6

| | | | Common descriptors (categorized) | | | | | |
|---|---|---|---|---|---|---|---|---|
| Average | Number of | | | | | | | Atom/ |
| number of | common | Comm. | Surface | Surface | Connectivity | Topology | Physical | bond |
| descriptors | descriptors | descr. % | property | area | indices | indices | property | counts |

Benzodiazepine receptor ligands

| | | | | | | | |
|---|---|---|---|---|---|---|---|
| 29.7 | 19 | 63.9% | 12 | 2 | 2 | 2 | 1 |

Serotonin receptor ligands

| | | | | | | | | |
|---|---|---|---|---|---|---|---|---|
| 32.7 | 16 | 48.9% | 7 | 1 | 2 | 2 | 2 | 2 |

Tyrosine kinase inhibitors

| | | | | | | | | |
|---|---|---|---|---|---|---|---|---|
| 19.7 | 15 | 76.1% | 5 | 2 | 2 | 1 | 3 | 2 |

Histamine H3 antagonists

| | | | | | | | |
|---|---|---|---|---|---|---|---|
| 38.7 | 13 | 33.6% | 6 | 1 | 3 | 1 | 2 |

Cyclooxygenase-2 inhibitors

| | | | | | | | |
|---|---|---|---|---|---|---|---|
| 31.3 | 13 | 41.5% | 6 | 1 | 2 | 1 | 3 |

As can be seen by the results above, common descriptors consistently occurred in all three simulations per activity class.

### EXAMPLE 2

Another example of the operation of the system 10 for performing virtual screening is provided below. In this example, the system 10 and the steps 100-120 and 200-250 are the same as described above, except as described herein. In this particular example, the system 10 operates to perform steps 100-120 and 200-250 as described above. The results are provided below from several “runs” (i.e., the operation of system 10) at step 210 where the processor 14 executes the instructions stored in the memory 18(2) which comprise the descriptor system 20 and the genetic algorithm system 32 to identify a set of suitable descriptors which will co-partition as many bait compounds 72 as possible. Table 7 below summarizes these results for the active 317 compounds used in this example:

TABLE 7
Top 10 Scoring Descriptor Sets from GA-MP on 21 Biological
Activity Classes

| Descriptors | nDS | Score | % P | P | nP | S | M | nM | ccₐᵥ |
|---|---|---|---|---|---|---|---|---|---|
| PEOE_VSA + 3, | 13 | 1.27 | 81.7 | 79 | 259 | 46 | 5 | 12 | 0.18 |
| PEOE_VSA − 3, | | | | | | | | | |
| PEOE_VSA − 5, RPC−, | | | | | | | | | |
| SMR_VSA0, | | | | | | | | | |
| SMR_VSA4, a_aro, | | | | | | | | | |
| a_nO, a_nS, b_ar, | | | | | | | | | |
| chilv_C, vdw_vol, | | | | | | | | | |
| vsa_don | | | | | | | | | |
| PEOE_VSA + 3, | 12 | 1.27 | 81.7 | 79 | 259 | 46 | 5 | 12 | 0.17 |
| PEOE_VSA − 3, | | | | | | | | | |
| PEOE_VSA − 5, RPC−, | | | | | | | | | |
| SMR_VSA0, | | | | | | | | | |
| SMR_VSA4, a_aro, | | | | | | | | | |
| a_n0, a_nS, chil v_C, | | | | | | | | | |
| vdw_vol, vsa_don | | | | | | | | | |
| PEOE_VSA + 3, | 12 | 1.27 | 81.7 | 79 | 259 | 46 | 5 | 12 | 0.17 |
| PEOE_VSA − 3, | | | | | | | | | |
| PEOE_VSA − 5, RPC+, | | | | | | | | | |
| SMR_VSA0, | | | | | | | | | |
| SMR_VSA4, a_aro, | | | | | | | | | |
| a_n0, a_nS, chilv_C, | | | | | | | | | |
| vdw_vol, vsa_don | | | | | | | | | |
| PEOE_VSA + 3, | 13 | 1.27 | 81.7 | 79 | 259 | 46 | 5 | 12 | 0.18 |
| PEOE_VSA − 3, | | | | | | | | | |
| PEOE_VSA − 5, RPC−, | | | | | | | | | |
| SMR_VSA0, | | | | | | | | | |
| SMR_VSA4, a_aro, | | | | | | | | | |
| a_n0, a_nS, b_ar, | | | | | | | | | |
| chilv_C, vdw_vol, | | | | | | | | | |
| vsa_don | | | | | | | | | |
| PEOE_VSA + 3, | 12 | 1.27 | 81.7 | 79 | 259 | 46 | 5 | 12 | 0.17 |
| PEOE_VSA − 3, | | | | | | | | | |
| PEOE_VSA − 5, RPC+, | | | | | | | | | |
| SMR_VSA0, | | | | | | | | | |
| SMR_VSA4, a_aro, | | | | | | | | | |
| a_n0, a_nS, chilv_C, | | | | | | | | | |
| vdw_vol, vsa_don | | | | | | | | | |
| PEOE_VSA + 3, | 12 | 1.27 | 81.7 | 82 | 259 | 48 | 4 | 10 | .017 |
| PEOE_VSA − 3, | | | | | | | | | |
| PEOE_VSA − 5, RPC−, | | | | | | | | | |
| SMR_VSA0, | | | | | | | | | |
| SMR_VSA4, a_aro, | | | | | | | | | |
| a_n0, a_nS, chil, | | | | | | | | | |
| chilv_C, vsa_don | | | | | | | | | |
| PEOE_VSA − 5, RPC−, | 12 | 1.26 | 81.4 | 73 | 258 | 42 | 7 | 17 | 0.23 |
| SMR_VSA0, | | | | | | | | | |
| SMR_VSA4, | | | | | | | | | |
| slogP_VSA1, VAdjMa, | | | | | | | | | |
| a_aro, a_n0, a_nS, | | | | | | | | | |
| b_lrotN, b_ar, vsa_don | | | | | | | | | |
| PEOE_VSA − 5, RPC−, | 11 | 1.26 | 81.4 | 73 | 258 | 42 | 7 | 17 | 0.23 |
| SMR_VSA0, | | | | | | | | | |
| SMR_VSA4, | | | | | | | | | |
| SlogP_VSA1, VAdjMa, | | | | | | | | | |
| a_aro, a_n0, a_nS, | | | | | | | | | |
| b_lrotN, vsa_don | | | | | | | | | |
| PEOE_VSA − 5, RPC+, | 12 | 1.26 | 81.4 | 73 | 258 | 42 | 7 | 17 | 0.23 |
| SMR_VSA0, | | | | | | | | | |
| SMR_VSA4, | | | | | | | | | |
| SlogP_VSA1, VAdjMa, | | | | | | | | | |
| a_aro, a_n0, a_nS, | | | | | | | | | |
| b_lrotN, b_ar, vsa_don | | | | | | | | | |
| PEOE_VSA − 5, RPC−, | 12 | 1.26 | 81.4 | 73 | 258 | 42 | 7 | 17 | 0.23 |
| SMR_VSA0, | | | | | | | | | |
| SMR_VSA4, | | | | | | | | | |
| SlogP_VSA1, VAdjMa, | | | | | | | | | |
| a_aro, a_n0, a_nS, | | | | | | | | | |
| b_lrotN, b_ar, vsa_don | | | | | | | | | |

| | | |
|---|---|---|
| a_aro, a_n0, a_nS, | 7 | consensus |
| PEOE_VSA − 5, | | |
| SMR_VSA0, | | |
| SMR_VSA4, vsa_don | | |

In Table 7: the “consensus” combination includes those descriptors that are shared among the top scoring combinations; “nDS” is the number of descriptors; “% P” is the percentage of active compounds in pure partitions; “P” is the number of pure partitions; “nP” is the total number of compounds in pure partitions; “S” is the number of singletons; “M” is the number of mixed partitions; “rnM” is the total number of compounds in mixed partitions; and ccₐᵥis the average pairwise descriptor correlation coefficient.

The present inventors found that overall classification accuracy of the system 10 was high with up to 81.7% of the compounds occurring in pure partitions. As a control, the processor 14 executed the instructions stored in the memory 18(2) which comprise the genetic algorithm system 32 to carry out 5,000 cycles with random descriptor settings and no score optimization. For these random predictions, an average score of 0.04 was obtained (as opposed to 1.27, the best score in Table 7), and only about 11.2% of the compounds were found in pure partitions. Between 11 and 13 descriptors were sufficient to achieve this level of accuracy, and the top scoring descriptor combinations were quite similar, having seven descriptors in common. Shared descriptors range from rather simple ones (e.g., counting the number of aromatic or oxygen atoms in a molecule) to fairly complex descriptors. Among classification errors, singletons (i.e., unassigned active compounds) were three to four times more frequent than molecules in mixed partitions (i.e., false positive recognitions).

Table 8 below shows results for corresponding calculations on the compound database 24 having about 2,000 background compounds (thought to be “inactive”), which increased the degree of difficulty for the classification of active molecules:

TABLE 8
Top 10 Scores on 21 Biological Activity Classes
in the Presence of 2000 Background Compounds

| Descriptors | nDS | Score | % P | P | nP | S | M | nM | ccₐᵥ |
|---|---|---|---|---|---|---|---|---|---|
| Kier3, PEOE_RPC− , | 19 | 0.50 | 63.1 | 69 | 200 | 86 | 22 | 31 | 0.23 |
| PEOE_VSA + 3, | | | | | | | | | |
| PEOE_VSA + 5, PEOE_VSA − 4, | | | | | | | | | |
| PEOE_VSA − 6, RPC−, | | | | | | | | | |
| SMR_VSA4, SLogP_VSA0, | | | | | | | | | |
| SlogP_VSA1, SLogP_VSA2, | | | | | | | | | |
| TPSA, VAdjMa, a_hyd, a_nN, | | | | | | | | | |
| a_nS, vsa_acc, vsa_pol, zagreb | | | | | | | | | |
| Kier3, PEOE_RPC−, | 18 | 0.50 | 62.8 | 67 | 199 | 74 | 28 | 44 | 0.24 |
| PEOE_VSA + 3, | | | | | | | | | |
| PEOE_VSA + 5, PEOE_VSA − 4, | | | | | | | | | |
| PEOE_VSA − 6, RPC−, | | | | | | | | | |
| SlogP_VSA0, SlogP_VSA1, | | | | | | | | | |
| SlogP_VSA2, TPSA, a_hyd, | | | | | | | | | |
| a_nN, a_nS, b_heavy, vsa_acc, | | | | | | | | | |
| vsa_pol, zagreb | | | | | | | | | |
| Kier3, PEOE_RPC−, | 18 | 0.50 | 62.8 | 67 | 199 | 74 | 28 | 44 | 0.24 |
| PEOE_VSA + 3, | | | | | | | | | |
| PEOE_VSA + 5, PEOE_VSA − 4, | | | | | | | | | |
| PEOE_VSA − 6, RPC− , | | | | | | | | | |
| SlogP_VSA0, SlogP_VSA1, | | | | | | | | | |
| SlogP_VSA2, TPSA, VAdjMa, | | | | | | | | | |
| a_hyd, a_nN, a_nS, vsa_acc, | | | | | | | | | |
| vsa_pol, zagreb | | | | | | | | | |
| Kier3, PEOE_RPC−, | 19 | 0.50 | 62.8 | 67 | 199 | 74 | 28 | 44 | 0.25 |
| PEOE_VSA + 3, | | | | | | | | | |
| PEOE_VSA + 5, PEOE_VSA − 4, | | | | | | | | | |
| PEOE_VSA − 6, RPC−, | | | | | | | | | |
| SlogP_VSA0, SlogP_VSA1, | | | | | | | | | |
| SlogP_VSA2, TPSA, VAdjMa, | | | | | | | | | |
| a_hyd, a_nN, a_nS, b_heavy, | | | | | | | | | |
| vsa_acc, vsa_pol, zagreb | | | | | | | | | |
| Kier3, PEOE_RPC−, | 19 | 0.49 | 62.5 | 67 | 198 | 78 | 25 | 41 | 0.25 |
| PEOE_VSA + 3, | | | | | | | | | |
| PEOE_VSA + 5, PEOE_VSA − 4, | | | | | | | | | |
| PEOE_VSA − 6, RPC−, | | | | | | | | | |
| SlogP_VSA0, SlogP_VSA1, | | | | | | | | | |
| SlogP_VSA2, TPSA, a_hyd, | | | | | | | | | |
| a_nN, a_nS, b_heavy, vsa_acc, | | | | | | | | | |
| vsa_pol, weinerPol, zagreb | | | | | | | | | |
| Kier3, PEOE_RPC−, | 19 | 0.49 | 62.5 | 67 | 198 | 78 | 25 | 41 | 0.25 |
| PEOE_VSA + 3, | | | | | | | | | |
| PEOE_VSA + 5, PEOE_VSA − 4, | | | | | | | | | |
| PEOE_VSA − 6, RPC−, | | | | | | | | | |
| SlogP_VSA0, SlogP_VSA1, | | | | | | | | | |
| SlogP_VSA2, TPSA, VAdjMa, | | | | | | | | | |
| a_hyd, a_nN, a_nS, vsa_acc, | | | | | | | | | |
| vsa_pol, weinerPol, zagreb | | | | | | | | | |
| Kier3, PEOE_RPC−, | 19 | 0.49 | 62.5 | 70 | 198 | 77 | 26 | 42 | 0.25 |
| PEOE_VSA + 3, | | | | | | | | | |
| PEOE_VSA + 5, PEOE_VSA − 4, | | | | | | | | | |
| PEOE_VSA − 6, RPC−, | | | | | | | | | |
| SlogP_VSA0, SlogP_VSA1, | | | | | | | | | |
| SlogP_VSA2, TPSA, VAdjMa, | | | | | | | | | |
| a_hyd, a_nN, a_nS, vsa_acc, | | | | | | | | | |
| vsa_other, vsa_pol, zagreb | | | | | | | | | |
| Kier3, PEOE_RPC−, | 20 | 0.49 | 62.5 | 70 | 198 | 77 | 26 | 42 | 0.27 |
| PEOE_VSA + 3, | | | | | | | | | |
| PEOE_VSA + 5, PEOE_VSA − 4, | | | | | | | | | |
| PEOE_VSA − 6, RPC−, | | | | | | | | | |
| SlogP_VSA0, SlogP_VSA1, | | | | | | | | | |
| SlogP_VSA2, TPSA, VAdjMa, | | | | | | | | | |
| a_hyd, a_nN, a_nS, b_heavy, | | | | | | | | | |
| vsa_acc, vsa_other, vsa_pol, | | | | | | | | | |
| zagreb | | | | | | | | | |
| Kier3, PEOE_RPC−, | 19 | 0.49 | 62.5 | 70 | 198 | 77 | 26 | 42 | 0.25 |
| PEOE_VSA + 3, | | | | | | | | | |
| PEOE_VSA + 5, PEOE_VSA − 4, | | | | | | | | | |
| PEOE_VSA − 6, RPC−, | | | | | | | | | |
| SlogP_VSA0, SlogP_VSA1, | | | | | | | | | |
| SlogP_VSA2, TPSA, a_hyd, | | | | | | | | | |
| a_nN, a_nS, b_heavy, vsa_acc, | | | | | | | | | |
| vsa_other, vsa_pol, zagreb | | | | | | | | | |
| Kier3, PEOE_RPC−, | 22 | 0.49 | 62.5 | 72 | 198 | 91 | 19 | 28 | 0.27 |
| PEOE_VSA + 3, | | | | | | | | | |
| PEOE_VSA + 5, PEOE_VSA − 4, | | | | | | | | | |
| PEOE_VSA − 6, RPC−, | | | | | | | | | |
| SMR_VSA4, SlogP_VSA0, | | | | | | | | | |
| SlogP_VSA1, SlogP_VSA2, | | | | | | | | | |
| TPSA, VAdjMa, a_hyd, a_nN, | | | | | | | | | |
| a_nS, b_heavy, vsa_acc, | | | | | | | | | |
| vsa_other, vsa_pol, weinerPol, | | | | | | | | | |
| zagreb | | | | | | | | | |

| | | |
|---|---|---|
| a_hyd, a_nN, a_nS, Kier3, | 17 | consensus |
| PEOE_RPC−, PEOE_VSA + 3, | | |
| PEOE_VSA + 5, PEOE_VSA − 4, | | |
| PEOE_VSA − 6, RPC−, | | |
| SLogP_VSA0, SLogP_VSA1, | | |
| SlogP_VSA2, TPSA, vsa_acc, | | |
| vsa_pol, zagreb | | |

Abbreviations for the terms used in Table 8 have been explained above in connection with Table 7. As to be expected, the scores and overall classification accuracy decreased, but approximately two-thirds of the active compounds were still correctly classified, with up to 63. 1% of active molecules occurring in pure partitions. In this case, for random predictions, an average score of 0.03 was obtained and a classification accuracy of 9.2%. Thus, the achieved enrichment of compounds with similar activity in unique partitions was still significant. For the expanded database, both the number of singletons and compounds in mixed partitions increased relative to the results obtained for the 21 activity classes only. However, among classification errors, the trend seen above in Table 7 reversed, and approximately twice as many compounds were found in mixed partitions than singletons. This can be rationalized by the significantly increased probability of obtaining mixed partitions in the presence of background compounds. As evident in Table 8, the number of descriptors among the top scoring combinations also increased with the number of database compounds, and 18 or 19 descriptors were required to achieve best performance. However, as seen before, the best descriptor combinations revealed in our calculations were also very similar in this case.

Having thus described the basic concept of the invention, it will be rather apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and scope of the invention. Further, the recited order of elements, steps or sequences, or the use of numbers, letters, or other designations therefor, is not intended to limit the claimed processes to any order except as may be explicitly specified in the claims. Accordingly, the invention is limited only by the following claims and equivalents thereto.

What is claimed is:

1. A method for identifying a small subgroup of compounds representative of a larger set of compounds, said method comprising:
providing a set of compounds;
obtaining one or more descriptor values for each compound in the set of compounds;
determining a median value for each of the descriptor values for the set of compounds;
partitioning the set of compounds into a plurality of partitions using each median value for the set of compounds; and
selecting compounds from each of the plurality of partitions to form a subgroup of compounds representative of the set of compounds.

2. The method as set forth in claim 1 further comprising:
repeating said obtaining, determining, and partitioning one or more times with different descriptor values than used previously.

3. The method as set forth in claim 1, wherein said partitioning the compounds into partitions comprises:
dividing the compounds into a first partition of compounds which have the descriptor value greater than the median value and a second partition which have the descriptor value less than the median value.

4. The method as set forth in claim 1, wherein said selecting comprises:
determining a partition median value for each of the descriptor values for the compounds within a partition; and
selecting from the partition one or more compounds that have each descriptor value being within a predetermined range of values away from a corresponding partition median value to represent the compounds within the partition.

5. The method as set forth in claim 1, wherein the descriptor values are descriptor types independently selected from the group consisting of chemical properties, structural properties, surface area properties, and electrochemical properties.

6. The method as set forth in claim 1, wherein the descriptor values are descriptor types independently selected from the group consisting of a sum of atomic polarizabilities of all atoms, a number of aromatic atoms, a number of H-bond donors, a number of heavy atoms, a number of hydrophobic atoms, a number of nitrogen atoms, a number of fluorine atoms, a number of sulfur atoms, a number of iodine atoms, a number of bonds between heavy atoms, a number of aromatic bonds, a number of double nonaromatic bonds, an atomic connectivity index (order 0), a carbon valence connectivity index (order 1), a carbon connectivity index (order 1), a greatest value in a distance matrix, a third kappa shape index, a relative negative partial charge, a total positive van der Waals surface area, a fractional negative polar van der Waals surface area, a fractional hydrophobic van der Waals surface area, a vertex adjacency information (magnitude), a vertex distance equality index, a vertex distance magnitude index, a sum of a van der Waals surface area of each of one or more atoms in each compound in the set of compounds, a van der Waals surface area calculated for a property of each compound selected from the group consisting of hydrogen-bond acceptor atoms, hydrogen-bond donor atoms, nondonor-acceptor atoms, and polar atoms, a van der Waals volume calculated using a connection table, and a Zagreb index.

7. The method as set forth in claim 1, wherein the descriptor values are different descriptor types that do not substantially correlate with each other.

8. The method as set forth in claim 1, further comprising:
choosing different types of descriptors to base the descriptor values on using a genetic algorithm.

9. The method as set forth in claim 8, wherein the different types of descriptors for the set of compounds each have value distributions from which the median values are calculated.

10. The method as set forth in claim 8 further comprising:
establishing an optimal combination of the different types of descriptors to base the descriptor values on using the genetic algorithm.

11. The method as set forth in claim 10, wherein a scoring function is used by the genetic algorithm during said establishing of the optimal combination of the different types of descriptors, the scoring function comprising:
$$s = \frac{100}{{N}_{total}} \times \frac{1}{( {N}_{total} - {N}_{P} ) + C / {C}_{act}} ,$$
wherein Nₜₒₜₐₗ is a first total number of active compounds in the set of compounds, Nₚ is a second total number of compounds in partitions which have one type of compound, C is a third total number of partitions which have one or more types of compounds, and C_{act} is a fourth total number of one or more activity classes present in the set of compounds.

12. The method as set forth in claim 1, wherein said obtaining one or more descriptor values comprises:
calculating the descriptor values using a molecular modeling program.

13. A computer-readable medium having stored thereon instructions for identifying a small subgroup of compounds representative of a larger set of compounds, which when executed by at least one processor, causes the processor to perform:
providing information representing a set of compounds;
obtaining one or more descriptor values for each compound in the set of compounds;
determining a median value for each of the descriptor values for the set of compounds;
partitioning the set of compounds into a plurality of partitions using each median value for the set of compounds; and
selecting compounds from each of the plurality of partitions to form a subgroup of compounds representative of the set of compounds.

14. The medium as set forth in claim 13 further comprising:
repeating said obtaining, determining, and partitioning one or more times with different descriptor values than used previously.

15. The medium as set forth in claim 13, wherein said partitioning the compounds into partitions comprises:
dividing the compounds into a first partition of compounds which have the descriptor value greater than the median value and a second partition which have the descriptor value less than the median value.

16. The medium as set forth in claim 13, wherein said selecting comprises:
determining a partition median value for each of the descriptor values for the compounds within a partition; and
selecting from the partition one or more compounds that have each descriptor value being within a predetermined range of values away from a corresponding partition median value to represent the compounds within the partition.

17. The medium as set forth in claim 13, wherein the descriptor values are descriptor types independently selected from the group consisting of chemical properties, structural properties, surface area properties, and electrochemical properties.

18. The medium as set forth in claim 13, wherein the descriptor values are descriptor types independently selected from the group consisting of a sum of atomic polarizabilities of all atoms, a number of aromatic atoms, a number of H-bond donors, a number of heavy atoms, a number of hydrophobic atoms, a number of nitrogen atoms, a number of fluorine atoms, a number of sulfur atoms, a number of iodine atoms, a number of bonds between heavy atoms, a number of aromatic bonds, a number of double nonaromatic bonds, an atomic connectivity index (order 0), a carbon valence connectivity index (order 1), a carbon connectivity index (order 1), a greatest value in a distance matrix, a third kappa shape index, a relative negative partial charge, a total positive van der Waals surface area, a fractional negative polar van der Waals surface area, a fractional hydrophobic van der Waals surface area, a vertex adjacency information (magnitude), a vertex distance equality index, a sum of a van der Waals surface area of each of one or more atoms in each compound in the set of compounds, a van der Waals surface area calculated for a property of each compound selected from the group consisting of hydrogen-bond acceptor atoms, hydrogen-bond donor atoms, nondonor-acceptor atoms, and polar atoms, a vertex distance magnitude index, a van der Waals volume calculated using a connection table, and a Zagreb index.

19. The medium as set forth in claim 13, wherein the descriptor values are different descriptor types that do not substantially correlate with each other.

20. The medium as set forth in claim 13 further comprising:
choosing different types of descriptors to base the descriptor values on using a genetic algorithm.

21. The medium as set forth in claim 20 wherein the different types of descriptors for the set of compounds each have value distributions from which the median values are calculated.

22. The medium as set forth in claim 20, further comprising:
establishing an optimal combination of the different types of descriptors to base the descriptor values on using the genetic algorithm.

23. The medium as set forth in claim 22, wherein a scoring function is used by the genetic algorithm during said establishing of the optimal combination of the different types of descriptors, the scoring function comprising:
$$s = \frac{100}{{N}_{total}} \times \frac{1}{( {N}_{total} - {N}_{P} ) + C / {C}_{act}} ,$$
wherein Nₜₒₜₐₗ is a first total number of active compounds in the set of compounds, Nₚ is a second total number of compounds in partitions which have one type of compound, C is a third total number of partitions which have one or more types of compounds, and C_{act} is a fourth total number of one or more activity classes present in the set of compounds.

24. The medium as set forth in claim 13, wherein said obtaining one or more descriptor values comprises:
calculating the descriptor values using a molecular modeling program.

25. A system for identifying a small group of compounds representative of a larger set of compounds, said system comprising:
a descriptor system that obtains one or more descriptor values for information representing each compound in the set of compounds;
a median determination system that determines a median value for each of the descriptor values for the set of compounds;
a partitioning system that partitions the set of compounds into a plurality of partitions using each median value for the set of compounds; and
a partition selection system that selects compounds from each of the plurality of partitions to form a subgroup representative of the set of compounds.

26. The system as set forth in claim 25, wherein the partition selection system causes operation of the descriptor system, the median determination system, and the partitioning system one or more times, the descriptor values each being a different type of descriptor than the descriptor values used previously.

27. The system as set forth in claim 25, wherein the partitioning system divides the compounds into a first partition of compounds which have the descriptor value greater than the median value and a second partition which have the descriptor value less than the median value.

28. The system as set forth in claim 25, wherein the partition selection system determines a partition median value for each of the descriptor values for the compounds within a partition and selects from the partition one or more compounds that have each descriptor value being within a predetermined range of values away from a corresponding partition median value to represent the compounds within the partition.

29. The system as set forth in claim 25, wherein the descriptor values are different descriptor types that do not substantially correlate with each other.

30. The system as set forth in claim 25, wherein the descriptor system chooses different types of descriptors to base the descriptor values on using a genetic algorithm.

31. The system as set forth in claim 30, wherein the different types of descriptors for the set of compounds each have value distributions from which the median values are calculated.

32. The system as set forth in claim 30, wherein the descriptor system establishes an optimal combination of the different types of descriptors to base the descriptor values on using the genetic algorithm.

33. The system as set forth in claim 32, wherein a scoring function is used by the genetic algorithm during establishment of the optimal combination of the different types of descriptors, the scoring function comprising:
$$s = \frac{100}{{N}_{total}} \times \frac{1}{( {N}_{total} - {N}_{P} ) + C / {C}_{act}} ,$$
wherein Nₜₒₜₐₗ is a first total number of active compounds in the set of compounds, Nₚ is a second total number of compounds in partitions which have one type of compound, C is a third total number of partitions which have one or more types of compounds, and C_{act} is a fourth total number of one or more activity classes present in the set of compounds.

34. The system as set forth in claim 25, wherein the descriptor system calculates the descriptor values using a molecular modeling program.

35. A method for virtual compound screening comprising:
combining a plurality of unidentified compounds with a plurality of bait compounds with known biological activities to create a set of compounds;
obtaining one or more descriptor values for each of the unidentified compounds and for each of the bait compounds in the set of compounds;
determining a median value for each of the descriptor values for the set of compounds;
partitioning the set of compounds into a plurality of partitions based on each median value;
recombining partitions which have at least two bait compounds to form a recombined set of compounds; and
selecting the recombined set of compounds for analysis of biological activity if an approximate target number of unidentified components remain in the recombined set of compounds.

36. The method as set forth in claim 35 further comprising:
repeating said obtaining, determining, partitioning and recombining with different descriptor values than used previously until the approximate target number of unidentified compounds remain in the recombined set of compounds.

37. The method as set forth in claim 36 further comprising:
reintroducing another set of bait compounds into the recombined set of compounds substantially prior to repeating said obtaining, the other set of bait compounds are identical to the bait compounds used during said combining.

38. The method as set forth in claim 35, wherein the target number of compounds is less than about 100 compounds.

39. The method as set forth in claim 35, wherein each bait compound comprises an active compound selected from the group consisting of benzodiazepine receptor ligands, serotonin receptor ligands, tyrosine kinase inhibitors, histamine H3 antagonists, cyclooxygenase-2 inhibitors, HIV protease inhibitors, carbonic anhydrase II inhibitors, β-lactamase inhibitors, protein kinase C inhibitors, estrogen antagonists, antihypertensive (ACE inhibitor), antiadrenergic (β-receptor), glucocorticoid analogues, angiotensin AT1 antagonists, aromatase inhibitors, DNA topoisomerase I inhibitors, dihydrofolate reductase inhibitors, factor Xa inhibitors, famesyl transferase inhibitors, matrix metalloproteinase inhibitors, and vitamin D analogues.

40. The method as set forth in claim 35, wherein each bait compound has a particular biological activity.

41. The method as set forth in claim 35, wherein said partitioning the compounds into partitions comprises:
dividing the compounds into a first partition of compounds which have the descriptor value greater than the median value and a second partition which have the descriptor value less than the median value.

42. The method as set forth in claim 35, wherein the descriptor values are different descriptor types independently selected from the group consisting of chemical properties, structural properties, surface area properties, and electrochemical properties.

43. The method as set forth in claim 35, wherein the descriptor values are descriptor types independently selected from the group consisting of a sum of atomic polarizabilities of all atoms, a number of aromatic atoms, a number of H-bond donors, a number of heavy atoms, a number of hydrophobic atoms, a number of nitrogen atoms, a number of fluorine atoms, a number of sulfur atoms, a number of iodine atoms, a number of bonds between heavy atoms, a number of aromatic bonds, a number of double nonaromatic bonds, an atomic connectivity index (order 0), a carbon valence connectivity index (order 1), a carbon connectivity index (order 1), a greatest value in a distance matrix, a third kappa shape index, a relative negative partial charge, a total positive van der Waals surface area, a fractional negative polar van der Waals surface area, a fractional hydrophobic van der Waals surface area, a vertex adjacency information (magnitude), a vertex distance equality index, a vertex distance-magnitude index, a sum of a van der Waals surface area of each of one or more atoms in each compound in the set of compounds, a van der Waals surface area calculated for a property of each compound selected from the group consisting of hydrogen-bond acceptor atoms, hydrogen-bond donor atoms, nondonor-acceptor atoms, and polar atoms, a van der Waals volume calculated using a connection table, and a Zagreb index.

44. The method as set forth in claim 35, wherein the descriptor values are different descriptor types that do not substantially correlate with each other.

45. The method as set forth in claim 35 further comprising:
choosing different types of descriptors to base the descriptor values on using a genetic algorithm.

46. The method as set forth in claim 45, wherein the different types of descriptors for the set of compounds each have value distributions from which the median values are calculated.

47. The method as set forth in claim 45 further comprising:
establishing an optimal combination of the different types of descriptors to base the descriptor values on using the genetic algorithm.

48. The method as set forth in claim 45, wherein a scoring function is used by the genetic algorithm during said establishing of the optimal combination of the different types of descriptors, the scoring function comprising:
S=Act(cp)×Pa(pop),
wherein Act(cp) is a first total number of co-partitioned known active compounds in the set of compounds and Pa(pop) is a second total number of populated partitions.

49. The method as set forth in claim 35, wherein said obtaining one or more descriptor values comprises:
calculating the descriptor values using a molecular modeling program.

50. A computer-readable medium having stored thereon instructions for virtual compound screening, which when executed by at least one processor, causes the processor to perform:
combining information representing a plurality of unidentified compounds with information representing a plurality of bait compounds with known biological activities to create a set of compounds;
obtaining one or more descriptor values for each of the unidentified compounds and for each of the bait compounds in the set of compounds;
determining a median value for each of the descriptor values for the set of compounds;
partitioning the set of compounds into a plurality of partitions based on each median value;
recombining partitions which have at least two bait compounds to form a recombined set of compounds; and
selecting the recombined set of compounds for analysis of biological activity if an approximate target number of unidentified compounds remain in the recombined set of compounds.

51. The medium as set forth in claim 50 comprising:
repeating said obtaining, determining, partitioning and recombining with different descriptor values than used previously until the approximate target number of unidentified compounds remain in the recombined set of compounds.

52. The medium as set forth in claim 51 further comprising:
reintroducing another set of bait compounds into the recombined set of compounds substantially prior to repeating said obtaining, the other set of bait compounds are identical to the bait compounds used during said combining.

53. The medium as set forth in claim 50, wherein the target number of compounds is less than about 100 compounds.

54. The medium as set forth in claim 50, wherein each bait compound comprises an active compound selected from the group consisting of benzodiazepine receptor ligands, serotonin receptor ligands, tyrosine kinase inhibitors, histamine H3 antagonists, cyclooxygenase-2 inhibitors, HIV protease inhibitors, carbonic anhydrase II inhibitors, β-lactamase inhibitors, protein kinase C inhibitors, estrogen antagonists, antihypertensive (ACE inhibitor), antiadrenergic (β-receptor), glucocorticoid analogues, angiotensin AT1 antagonists, aromatase inhibitors, DNA topoisomerase I inhibitors, dihydrofolate reductase inhibitors, factor Xa inhibitors, famesyl transferase inhibitors, matrix metalloproteinase inhibitors, and vitamin D analogues.

55. The medium as set forth in claim 50, wherein each bait compound has a particular biological activity.

56. The medium as set forth in claim 50, wherein said partitioning the compounds into partitions comprises:
dividing the compounds into a first partition of compounds which have the descriptor value greater than the median value and a second partition which have the descriptor value less than the median value.

57. The medium as set forth in claim 50, wherein the descriptor values are descriptor types independently selected from the group consisting of chemical properties, structural properties, surface area properties, and electrochemical properties.

58. The medium as set forth in claim 50, wherein the descriptor values are descriptor types independently selected from the group consisting of a sum of atomic polarizabilities of all atoms, a number of aromatic atoms, a number of H-bond donors, a number of heavy atoms, a number of hydrophobic atoms, a number of nitrogen atoms, a number of fluorine atoms, a number of sulfur atoms, a number of iodine atoms, a number of bonds between heavy atoms, a number of aromatic bonds, a number of double nonaromatic bonds, an atomic connectivity index (order 0), a carbon valence connectivity index (order 1), a carbon connectivity index (order 1), a greatest value in a distance matrix, a third kappa shape index, a relative negative partial charge, a total positive van der Waals surface area, a fractional negative polar van der Waals surface area, a fractional hydrophobic van der Waals surface area, a vertex adjacency information (magnitude), a vertex distance equality index, a vertex distance magnitude index, a sum of a van der Waals surface area of each of one or more atoms in each compound in the set of compounds, a van der Waals surface area calculated for a property of each compound selected from the group consisting of hydrogen-bond acceptor atoms, hydrogen-bond donor atoms, nondonor-acceptor atoms, and polar atoms, a van der Waals volume calculated using a connection table, and a Zagreb index.

59. The medium as set forth in claim 50, wherein the descriptor values are different descriptor types that do not substantially correlate with each other.

60. The medium as set forth in claim 50 further comprising:
choosing different types of descriptors to base the descriptor values on using a genetic algorithm.

61. The medium as set forth in claim 60, wherein the different types of descriptors for the set of compounds each have value distributions from which the median values are calculated.

62. The medium as set forth in claim 60 further comprising:
establishing an optimal combination of the different types of descriptors to base the descriptor values on using the genetic algorithm.

63. The medium as set forth in claim 62, wherein a scoring function is used by the genetic algorithm during said establishing of the optimal combination of the different types of descriptors, the scoring function comprising:
S=Act(cp)×Pa(pop),
wherein Act(cp) is a first total number of co-partitioned known active compounds in the set of compounds and Pa(pop) is a second total number of populated partitions.

64. The medium as set forth in claim 50, wherein said obtaining one or more descriptor values comprises:
calculating the descriptor values using a molecular modeling program.

65. A system for virtual compound screening comprising:
a bait compound system that combines information representing a plurality of unidentified compounds with information representing a plurality of bait compounds with known biological activities to form a set of compounds;
a descriptor system that obtains one or more descriptor values for each of the unidentified compounds and for each of the bait compounds in the set of compounds;
a median determination system that determines a median value for each of the descriptor values for the set of compounds;
a partitioning system that partitions the set of compounds into a plurality of partitions based on each median value;
a partition recombination system that recombines partitions which have at least two bait compounds to form a recombined set of compounds; and
a compound selection system that selects the recombined set of compounds for analysis of biological activity if an approximate target number of unidentified compounds remain in the recombined set of compounds.

66. The system as set forth in claim 65, wherein the compound selection system causes operation of the descriptor system, the median determination system, the partitioning system, and the partition recombination system with different descriptor values than used previously until the approximate target number of unidentified compounds remain in the recombined set of compounds.

67. The system as set forth in claim 66, wherein the compound selection system causes another set of bait compounds to be reintroduced into the recombined set of compounds substantially prior to operation of the descriptor system, the other set of bait compounds being identical to the bait compounds used by the bait compound system.

68. The system as set forth in claim 65, wherein the partitioning system divides the compounds into a first partition of compounds which have the descriptor value greater than the median value and a second partition which have the descriptor value less than the median value.

69. The system as set forth in claim 65, wherein the descriptor values are different descriptor types that do not substantially correlate with each other.

70. The system as set forth in claim 65, wherein the descriptor system chooses different types of descriptors to base the descriptor values on using a genetic algorithm.

71. The system as set forth in claim 70, wherein the different types of descriptors for the set of compounds each have value distributions from which the median values are calculated.

72. The system as set forth in claim 70, wherein the descriptor system establishes an optimal combination of the different types of descriptors to base the descriptor values on using the genetic algorithm.

73. The system as set forth in claim 72, wherein a scoring function is used by the genetic algorithm during establishment of the optimal combination of the different types of descriptors, the scoring function comprising:
S=Act(cp)×Pa(pop),
wherein Act(cp) is a first total number of co-partitioned known active compounds in the set of compounds and Pa(pop) is a second total number of populated partitions.

74. The system as set forth in claim 65, wherein the descriptor system calculates the descriptor values using a molecular modeling program.
